(19) United States
(12) Patent Application Publication
(10) Pub. No.: US 2018/0256690 A1
(43) Pub. Date: Sep. 13, 2018

(54) PROTECTION AND SEALING OF THE OCULAR SURFACE BARRIER BY CLUSTERIN

(71) Applicant: University of Southern California, Los Angeles, CA (US)

(72) Inventors: Elizabeth M. Fini, Pasadena, CA (US); Shinwu Jeong, Buena Park, CA (US)

(21) Appl. No.: 15/759,487

(22) PCT Filed: Sep. 15, 2016
(86) PCT No.: PCT/US16/52002

§ 371 Date: Mar. 12, 2018

Related U.S. Application Data
(60) Provisional application No. 62/219,018, filed on Sep. 15, 2015.

(51) Int. Cl. A61K 38/57; A61K 38/17; A61K 9/00; A61K 9/08; C07K 14/00

(52) U.S. Cl. CPC A61K 38/57; A61K 38/1709; C07K 14/00; A61K 9/08; A61K 9/0048

(57) ABSTRACT

A method of treating dry eye disease is provided. The method includes administering to a patient in need thereof an effective amount of a pharmaceutical composition that includes an isolated clusterin or an isolated protein substantially the same as clusterin. An amount of the pharmaceutical composition immediately below the effective amount of the pharmaceutical composition has substantially no beneficial effect in treating dry eye disease.

### RELATED APPLICATIONS

This application claims the benefit of U.S. Provisional Application No. 62/219,018, filed Sep. 15, 2015, the entire contents of which are incorporated herein by reference in its entirety.

### FIELD OF THE INVENTION

The present invention relates in general to pharmaceutical compositions comprising clusterin or polypeptides substantially the same as clusterin and to treatment methods for dry eye disease.

### BACKGROUND OF THE INVENTION

The ocular surface is directly exposed to the outside environment, where it is subject to desiccation and interaction with noxious agents, thus it must function as a barrier to protect the underlying tissue [1]. Membrane-associated mucins project from the apical cell layer of the corneal and conjunctival epithelia into the tear film, where they bind multiple oligomers of the lectin LGALS3 to form a highly organized glycocalyx, creating the transcellular barrier [2, 3]. In addition, tight junctions seal the space between adjacent cells to create the paracellular barrier [4]. The barriers appear to be functionally linked via the cytoskeleton [5].

Ocular surface barrier disruption is a sign of dry eye, a disorder caused by inadequate hydration by the tears, which results in discomfort, affects quality of vision, and can cause blindness [6]. Dry eye affects ˜5 million people over the age of 50 in the USA (especially women) and almost 15% of the population at all ages, comprising upwards of 30-40 million people [7]. In all forms of dry eye, reduced tear flow and/or increased evaporation leads to tear hyperosmolarity, initiating the vicious circle of dry eye pathology. Hyperosmolarity induces inflammatory cascade activation [8-10], promotes apoptosis [11-13], and stimulates expression and activity of matrix metalloproteinases (MMPs) [14, 15], leading to ocular surface barrier disruption [16]. Disruption of the ocular surface barrier is assessed clinically by measuring uptake of water-soluble dyes such as rose-bengal, lissamine green or fluorescein, which occurs in a distinctive punctate pattern in dry eye [17, 18]. The normal ocular surface exhibits variable levels of dye uptake, possibly reflecting the natural processes of cellular desquamation and shedding of mucin ectodomains [1, 18, 19]. Higher levels of dye uptake are diagnostic of dry eye, however mechanisms are not fully defined [18, 20, 21].

MMP9 is recognized as a causal mediator of ocular surface barrier disruption due to desiccating stress in both mice [14, 15], and humans [22]. To help generate hypotheses about mechanisms of dry eye, we performed a yeast two-hybrid screen for corneal proteins that interact with MMP9 [23]. A single candidate was validated: clusterin (CLU). Functional studies revealed that CLU is a potent inhibitor of MMP9 enzymatic activity, as well as activity of other MMPs. When CLU was added to confluent epithelial cell cultures treated with MMP9, tight junctions were protected against MMP9 proteolysis [23].

Human CLU is secreted as a 62-kDa glycoprotein (with an apparent mass of 70-80 kDa as evaluated by denaturing SDS-PAGE) composed of two disulfide-bonded polypeptide chains derived from proteolytic cleavage of an intracellular precursor [24]. With three sites for N-linked glycosylation on each chain, secreted CLU is 17-27% N-linked carbohydrate by weight [25]. Three long natively disordered regions linked to amphipathic helices form a dynamic, molten globule-like binding site, providing the ability to interact with a variety of molecules [26]. Also known as apolipoprotein J or ApoJ, CLU associates with discrete subclasses of high-density lipoproteins [27]. CLU is cytoprotective [28, 29] and anti-inflammatory [30], and it also functions as an extracellular molecular chaperone, acting to maintain proteostasis by inhibiting the aggregation of stress-induced misfolded proteins and facilitating their clearance from extracellular fluids [31, 32]. Consistent with this, the only known phenotype of CLU knockout mice maintained under unstressed conditions is the gradual accumulation of insoluble protein deposits in the kidney [33]. On the other hand, CLU knockout mice exhibit distinct phenotypes when conditions are created to model inflammatory diseases [30, 34].

CLU is found in bodily fluids and is expressed prominently by epithelia at fluid-tissue interfaces [35, 36]. In the context of its known properties, this expression pattern suggests that CLU protects barrier cells from the environment. With regard to the ocular surface-tear interface, CLU was identified as the most abundant transcript in the human corneal epithelium [37]. CLU is expressed in the apical corneal epithelial cell layers in both human [38] and mouse [23], and has also been identified in human tears [39-41]. Expression of CLU in the ocular surface epithelia is dramatically reduced in human inflammatory disorders that manifest as severe dry eye [38]. Similarly, we showed recently that both CLU protein and mRNA levels in the ocular surface epithelia are reduced by ˜30% when desiccating stress is induced in a preclinical mouse model for dry eye [23]. In addition, a striking reduction of CLU expression was observed in cultured human corneal epithelial cells treated with inflammatory mediators [23]. Collectively, these results suggest that down-regulation of CLU expression at the ocular surface subjected to desiccating stress in dry eye is due to activation of the inflammatory cascade.

### SUMMARY OF THE INVENTION

One aspect of the present invention is directed to a method of treating dry eye disease. The method includes administering to a patient in need thereof an effective amount of a pharmaceutical composition that includes an isolated clusterin or an isolated protein substantially the same as clusterin. An amount of the pharmaceutical composition immediately below the effective amount of the pharmaceutical composition has substantially no beneficial effect in treating dry eye disease.

In one embodiment, the pharmaceutical composition includes a secreted clusterin.

In another embodiment, the administration is topical.

In another embodiment, the pharmaceutical composition further includes a liquid carrier, and administration is by contacting the pharmaceutical composition to the surface of an eye of the patient.

In another embodiment, the pharmaceutical composition further includes a carrier.

In another embodiment, the carrier is a sterile solution.

In another embodiment, the clusterin is recombinant human clusterin.

Another aspect of the present invention is directed to a method of sealing and protecting the ocular surface barrier. The method includes administering to a patient in need thereof an effective amount of a pharmaceutical composition that includes an isolated clusterin or an isolated protein substantially the same as clusterin. An amount of the pharmaceutical composition immediately below the effective amount of the pharmaceutical composition has substantially no beneficial effect in sealing the ocular surface barrier.

In some embodiments, “immediately below” can represent an amount within 99% of the effective amount. In other embodiments “immediately below” can represent an amount within 95% of the effective amount, within 90% of the effective amount, within 80% of the effective amount, within 70% of the effective amount, or within 60% of the effective amount.

In one embodiment, the pharmaceutical composition includes a secreted clusterin.

In another embodiment, the administration is topical.

In another embodiment, the pharmaceutical composition further includes a liquid carrier, and administration is by contacting the pharmaceutical composition to the surface of an eye of the patient.

In another embodiment, the pharmaceutical composition further includes a carrier.

In another embodiment, the carrier is a sterile solution.

In another embodiment, the clusterin is recombinant human clusterin.

The present invention is based on the hypothesis that reduced levels of clusterin (CLU) result in vulnerability to barrier disruption using the preclinical mouse model.

The present invention discloses what can be referred to as the “sealing and healing” of the ocular surface barrier. The clusterin pharmaceutical compositions disclosed herein, when dosed and administered according to the present invention binds to the ocular surface, as shown for instance by an imaging assay (confocal), and seals the barrier, as shown for instance by a functional assay (fluorescein staining).

Concurrently, the clusterin pharmaceutical compositions disclosed, when dosed and administered according to the present invention, also protect, and thus promote healing, as determined by biochemical assay (apoptosis assay and western blotting). In this way, the clusterin pharmaceutical compositions are anti-apoptotic and proteostatic. These protective properties achieved when dosed and administered have never been previously demonstrated at the ocular surface in dry eye. Thus the preset clusterin pharmaceutical composition, used at the right dose, “ameliorates” ocular surface disease in dry eye, i.e., barrier disruption.

One important point about clusterin when administered according to the present invention is that it binds the ocular surface. Drug delivery at the ocular surface is usually a problem, as the drug is washed out of the eye quickly by tears. In contrast, the pharmaceutical compositions of the present invention are retained at the ocular surface for at least two hours (and probably much longer), as determined by continued sealing.

The clusterin composition disclosed herein appear to coat the ocular surface in areas where barrier disruption has occurred. Thus, in some embodiments, the clusterin composition action is analogous to caulking or plastering of cracks in a wall. In some cases, the clusterin composition can be thought of as a therapeutic “plaster” or “bandage”. At the same time, the clusterin compositions are protective, promoting the ability of the ocular surface to reconstitute itself, i.e., heal.

Other aspects and advantages of the invention will be apparent from the following description and the appended claims.

### BRIEF DESCRIPTION OF THE DRAWINGS

FIG. 1: Topical CLU protects the ocular surface barrier against functional disruption by desiccating stress. The standard desiccating stress (DS) protocol was applied, while eyes were left untreated (UT) or treated topically 4 times/day with 1 μL of CLU formulated in PBS, or with PBS control. Non-stressed (NS) mice housed under normal ambient conditions served as a baseline control. After the indicated time period, barrier integrity was assayed by measuring corneal epithelial uptake of fluorescein (FU=Fluorescence Units at 521 nm). Values are expressed as the mean±SD. (A) The desiccating stress (DS) protocol was applied for 5 days while also treating with rhCLU at 10 or 100 μg/mL. *P<0.0001 (n=9). (B) The desiccating stress (DS) protocol was applied for 7 days while also treating with rhCLU at 1 or 10 μg/mL. *P<0.0001 (n=4). (C) The desiccating stress (DS) protocol was applied for 5 days while also treating with human plasma CLU (pCLU) at 2 μg/mL *P<0.0001 (n=4). (D) The desiccating stress (DS) protocol was applied for 5 days while also treating with recombinant mouse CLU (rmCLU) at 2 μg/mL. *P<0.0001 (n=4)

FIG. 2: Topical CLU protects the ocular surface barrier via an all-or-none mechanism. The standard desiccating stress (DS) protocol was applied, while eyes were left untreated (UT) or treated topically 4 times/day with 1 μL of CLU formulated in PBS, or with PBS control. Non-stressed (NS) mice housed under normal ambient conditions served as a baseline control. After the indicated time period, barrier integrity was assayed by measuring corneal epithelial uptake of fluorescein (FU=Fluorescence Units at 521 nm). Values are expressed as the mean±SD. (A) Dose response experiment. The desiccating stress (DS) protocol was applied for 5 days while also treating with (Left) recombinant human CLU (rhCLU) at the indicated 10-fold dilutions (n=6), (Middle) recombinant human CLU (rhCLU) at 0.1, 0.3, 0.6, or 1 μg/mL (n=6), or (Right) recombinant mouse CLU (rmCLU) at 0.3, 0.6, and 1 μg/mL (n=4). *P<0.0001 (B) Experiment comparing CLU with BSA. The desiccating stress (DS) protocol was applied for 5 days while also treating with recombinant human CLU (rhCLU) and BSA, individually or in combination, as indicated. *P<0.0001 (n=4) (C) Stress reduction experiment. The standard desiccating stress (DS) protocol was applied for 5 days while eyes were also treated with recombinant human CLU (rhCLU) at 0.01, 0.1, and 1 μg/mL. Using a subset (n=4) of each treatment group the effect of each rhCLU dose on integrity of the ocular surface barrier was confirmed by the fluorescein uptake test at day 5. Then the rest of the mice in each treatment group were subjected for two more days to a more moderate desiccating stress by continuing with the air draft and heat, but omitting scopolamine and CLU treatments. The fluorescein uptake test was then performed on these remaining mice. *P=0.004 (n=4); **P=0.05 (n=4)

FIG. 3: Topical CLU ameliorates pre-existing ocular surface barrier disruption caused by desiccating stress. (Left) The standard desiccating stress (DS) protocol was applied for 5-days to create ocular surface disruption. Non-stressed (NS) mice housed under normal ambient conditions served as a baseline control. (Left) After the indicated time period, barrier disruption was confirmed by measuring corneal epithelial uptake of fluorescein (FU=Fluorescence Units at 521 nm) in a subset of mice. Values are expressed as the mean±SD. *p<0.0001 (n=4) (Right) The same desiccating stress (DS) protocol was continued for another 5 days while eyes with desiccating stress were treated topically with 1 μL of recombinant human CLU (rhCLU) formulated in PBS at 2 μg/mL, or with PBS control, 4 times/day. The fluorescein uptake test was then performed on these remaining mice. Values are expressed as the mean±SD. *p<0.0001(n=4).

FIG. 4: Topical CLU directly seals the ocular surface barrier disrupted by desiccating stress. The standard desiccating stress (DS) protocol was applied for 5-days to create ocular surface disruption. Non-stressed (NS) mice housed under normal ambient conditions served as a baseline control. Eyes with desiccating stress were then treated topically, a single time, with 1 μL of CLU formulated in PBS, 1 μL of BSA formulated in PBS for comparison, or 1 μL of PBS control. Barrier disruption was assayed by measuring corneal epithelial uptake of fluorescein (FU=Fluorescence Units at 521 nm). Values are expressed as the mean±SD. (A) Eyes were treated a single time with recombinant human CLU (rhCLU) at 1, 3, 6 or 10 μg/mL, BSA at 10 μg/mL, or PBS. Fifteen minutes later, the fluorescein uptake test was performed, before there was time for barrier repair to occur. *P<0.0001 (n=4). (B) Images of central cornea from the experiment shown in (A), obtained using laser scanning confocal microscopy at 10× magnification. One representative image out of two independent experiments is shown. Scale bar=100 μm. (C) Eyes were treated a single time with recombinant human CLU (rhCLU) at 10 μg/mL (right eyes) or PBS (left eyes). Then the mice were kept further for 2 h or 16 h while continuing with the same desiccating stress protocol. The fluorescein uptake test was performed following the indicated time period to assess the time length of CLU treatment effect. *p<0.0001 (n=4)

FIG. 5: Topical CLU binds selectively to the ocular surface subjected to desiccating stress, and to LGALS3 in vitro. (A) The standard desiccating stress (DS) protocol was applied for 5-days to create ocular surface disruption. Non-stressed (NS) mice housed under normal ambient conditions were included for comparison. Eyes were treated with CF-594-anti-His antibody that binds to the His tag of recombinant human CLU (rhCLU), or with a complex of the antibody-rhCLU, for 15 min, followed by confocal imaging of central cornea. Images were taken at 10× magnification. Scale bar=100 μm. (B) A DS eye was treated with a complex of the antibody-rhCLU (red) as in (A), as well as a fluorescent membrane tracer DiO (green). Images were taken at 20× magnification. In the left panel only CLU was projected. The right three panels show one Z-section plane with cross-sections oriented to the XY, YZ, and XZ axes, generated using Image J software. Yellow indicates regions of co-localization of the red and green signal. Scale bar=100 μm. (C) LGALS3-Sepharose affinity column chromatography. 1.5 μg rhCLU was applied to a 300 μL LGALS3 affinity column equilibrated in PBS containing 0.1% Triton X-100 (PBST) and the column was washed with PBST. To test sugar-binding specificity, the column was then treated sequentially with a non-competing disaccharide, sucrose (0.1 M), and then a competing disaccharide, 0.1 M lactose, dissolved in PBST. Western blotting was used to quantify CLU in the resulting fractions. Loading of the “Lac” lane represents a 1:10 dilution of the input and the “Beads” lane is a 1:4 dilution of the input, thus ˜2.5× more CLU was Lac-eluted than retained on the beads. FT=flow-through; Suc=sucrose; Lac=lactose

FIG. 6: Topical CLU protects the ocular surface barrier against proteolytic damage due to desiccating stress. (A) The standard desiccating stress (DS) protocol was applied, while eyes were left untreated (UT) or treated topically, 4 times/day, with 1 μL of recombinant human CLU (rhCLU) formulated in PBS, or with 1 μL of PBS control. Non-stressed (NS) mice housed under normal ambient conditions were included as a control for PBS treatment. At the end of the experiment, eyes were removed and embedded for frozen sectioning at 10 μm thickness. TUNEL staining was performed and nuclei were counterstained with DAPI. Images were taken at 20× magnification. Arrows indicate apoptotic cells in the apical ocular surface epithelium of DS+PBS eyes. (B) The standard desiccating stress (DS) protocol was applied, while eyes were left untreated (UT) or treated topically, 4 times/day, with 1 μL of recombinant human CLU (rhCLU) formulated in PBS, or with 1 μL of PBS control. Non-stressed (NS) mice housed under normal ambient conditions were included as a control for PBS treatment. Desiccating stress was applied to 7 mice per treatment group for 5 days (OCLN) or 9 days (LGALS3) while treated with PBS or CLU at 1 μg/mL. Then total proteins were extracted from the ocular surface epithelia using TRIzol, pooled among the same treatment groups, and subjected to Western blotting with anti-LGALS3 and anti-OCLN antibodies. The protein band image was obtained by Fuji Doc digital camera. “F” indicates full length LGALS3 protein, and “C” is the cleaved product of LGALS3. A digital image analyzer built into the camera was used to quantify the density of individual protein bands. The relative cleavage of LGALS3 was calculated by ratio of the C over the total (F+C) LGALS3 protein. The relative amount of OCLN was normalized to the loading control (ACTB) in each gel lane. (C) Stratified HCLE cells were treated with TNFA (5 ng/mL), alone or with recombinant human CLU (rhCLU) (4 μg/mL) or BSA (40 μg/mL) for 24 h. The conditioned media were subject to gelatin zymography and the developed MMP9 image were analyzed by Image J software. *P<0.05 (n=3, student's t-test)

FIG. 7: Causal association between endogenous CLU concentration in tears and ocular surface barrier vulnerability. (A) Tears were collected from mice housed under normal ambient conditions or after application of the standard desiccating stress (DS) protocol for 5-days, and ELISA was used to measure CLU concentration (*P=5×10⁻⁸ n=6, student's t-test). (B) Representative transmission electron microscopy comparing images of anterior cornea from wild type C57BL6/J mice (A and C) and mice with homozygous CLU^{-/-} knockout on the C57BL6/J background (B and D). In low power (4000×) magnifications (A and B), five layers of epithelial cells divided into squamous, wing, and basal cell regions are visualized along with an intact basement membrane and Bowman's layer in both types of animals. Higher power images (C and D, 20,000×) of similar regions to those boxed in panels A and B show numerous surface microplicae (fat arrows) in both genotypes. Desmosomes (thin arrows) are similar in both frequency and structure. Higher power images (not shown) demonstrate intact adherens junctions in both genotypes. (C) Tears from wild type or heterozygous CLU^{+/-} knockout mice kept at ambient conditions were collected and ELISA was used to measure CLU concentration (p=2.1×10⁻⁵; n=7, student's t-test). (D) Wild type mice or heterozygous CLU^{+/-} knockout mice were subjected to the standard desiccating stress protocol, but without scopolamine injection for four weeks and then ocular surface barrier integrity was measured by fluorescein uptake (**p<0.0001, n=4).

FIG. 8: Co-localization of fluorescein and CLU on the OCS after 5-day DS. Rhodamine labeled hrCLU was prepared according to Bauskar et al., 2015 PLOS One. Fluorescein and CLU were mixed (2 μl CLU+1 μl fluorescein) and then 2 μl of the resulting mixture instilled to the OCS in vivo for 15 min before removing the eye to take the confocal image, as described in Bauskar et al., 2015. Corneas were imaged by LSCM with simultaneous 2-color excitation and detection performed at 10× magnification. Fluorescein and CLU were distinguished by the color of fluorescence emission (far-red for CLU, green for fluorescein). The 3 rows in the figure represent 3 different corneas. Bar=100 CLU binds selectively to the ocular surface subjected to desiccating stress in regions of barrier disruption.

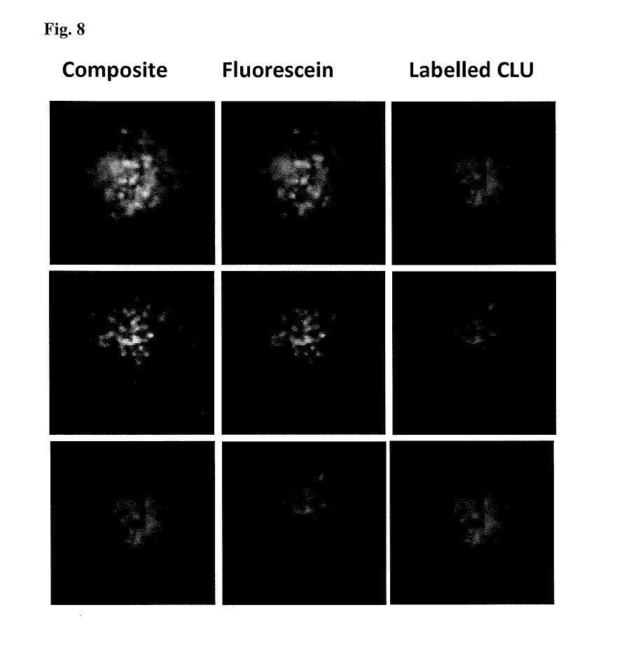

FIG. 9: Predicted human CLU structure. Schematic adapted from (Wilson and Easterbrook-Smith, 2000; Jones and Jomary, 2002; Bailey et al., 2001). The 22-mer secretory signal peptide is proteolytically cleaved from the 449 amino acid precursor polypeptide chain and subsequently the chain is cleaved again between residues Arg227-Ser228 to generate an α-chain and a β-chain. These are assembled in anti-parallel fashion to generate a heterodimeric molecule in which the cysteine-rich centers (red boxes) are linked by five disulfide bridges (black lines) and flanked by five predicted amphipathic α-helices (yellow ovals). The six sites for N-linked glycosylation are indicated (white spots). Amino acid numbering for the N- and C-termini, the cleavage sites, and the sites for N-linked glycosylation are indicated, as in (Kapron et al., 1997). Cysteines in alpha chain at positions: 102, 113, 116, 121, 129; N-glycosylation sites at positions: 86, 103, 145; Cysteines in beta chain at positions: 313, 305, 302, 295, 285; N-glycosylation sites at positions: 374, 354, 291.

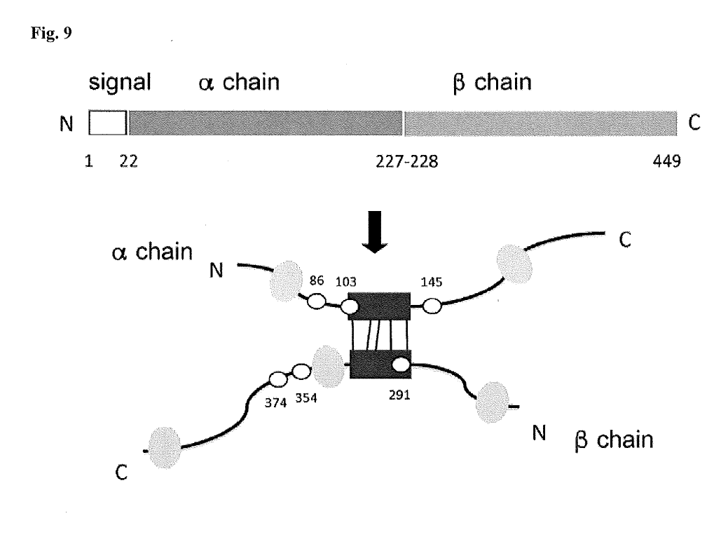

FIG. 10: Conceptual model depicting CLU binding to areas of barrier disruption at the ocular surface subjected to desiccating stress. Membrane-Associated Mucins (fuscia, dark blue and gold), LGALS3 (green) and CLU (dark blue with blue and coral “antlers”) are shown interacting with one another, and with the lipid bilayer of the apical epithelial cells (light blue), in this artist's conception of the ocular surface. Membrane-Associated Mucins are depicted as long, flexible rods (fuscia) traversing the lipid bilayer of the apical epithelial cells of the ocular surface, with their intracellular domains projecting into the cytoplasm (blue). The carbohydrate chains (gold) linked to the extracellular domains are extensively branched. Following exposure to desiccating stress, membrane-associated mucins may be proteolytically cleaved, leaving membrane-embedded protein “stubs” (fuscia). LGALS3 molecules (green) are shown with the C-terminal carbohydrate-binding domain appearing as a “mouth” linked to the N-terminal multimerization domain by a long thread. Some of these LGALS3 molecules are depicted as self-associating via their multimerization domains, a requirement for network formation and exclusion of clinical dyes. In other cases, the multimerization domain is drawn as proteolytically cleaved, leaving only the carbohydrate-binding domain. CLU molecules (blue) are schematically modeled after a milking stool. The “seat” of the stool represents the disulfide-bonded region of the polypeptide chains decorated by carbohydrate chains (blue and coral) emanating from six attachment sites. The three legs of the stool represent the C-terminal and N-terminal portions of the molecule containing the amphipathic helices. The “arm” of the stool is the C-terminal portion lacking an amphipathic helix. Galactose moieties on both the mucin and CLU carbohydrate chains are depicted as small “marbles” (yellow). The carbohydrate-binding domains (“mouths”) of LGALS3 molecules are shown binding to (“eating”) the yellow globes. CLU molecules are shown in various interactions 1) self-associating, 2) binding to the lipid bilayer, and 3) associating with proteolyzed mucin “stubs”. In the foreground, the proteolytically cleaved carbohydrate-binding domain of an LGALS3 molecule is shown binding to a marble on a carbohydrate chain of a CLU molecule. This drawing aims to illustrate the idea that all-or-none sealing of the ocular surface barrier disrupted by desiccating stress occurs when the concentration of CLU molecules is high enough to compete effectively with mucins for binding to LGALS3 molecules.

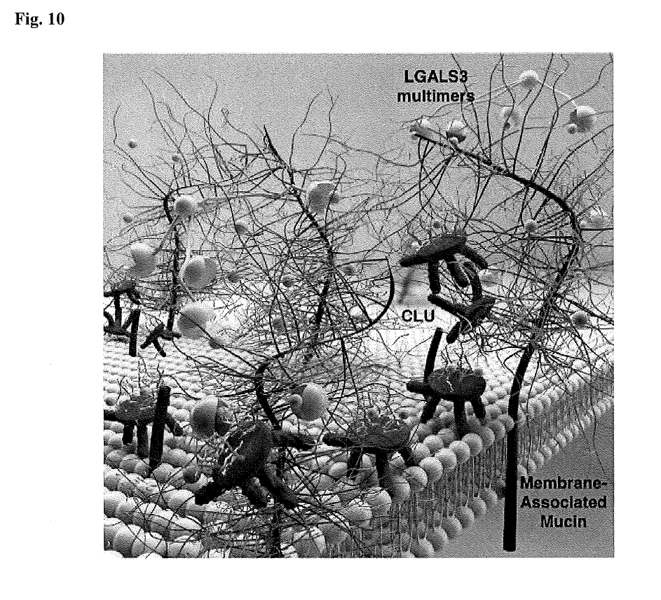

### DETAILED DESCRIPTION OF THE INVENTION DEFINITIONS

Unless otherwise indicated, all terms used herein have the meanings given below, and are generally consistent with same meaning that the terms have to those skilled in the art of the present invention. It is to be understood that this invention is not limited to the particular methodology, protocols, and reagents described, as these may vary.

The term “clusterin” refers to human clusterin, including secreted clusterin and nuclear clusterin, or any subunit, fragment or region of either capable of preventing uptake of clinical fluorescein dye. The term clusterin optionally encompasses non-peptidic components, such as carbohydrate groups or any other non-peptidic substituents that may be added to clusterin by a cell in which the protein is produced, and may vary with the type of cell. Clusterin can also include synthetic peptides. A His tag can be added to the end of a protein.

The terms “treatment” or “treating” refers to both therapeutic treatment and prophylactic or preventative measures, wherein the object is to prevent or slow down (lessen) the targeted pathologic condition or disorder. It may also encompass relief of symptoms associated with a pathological condition or disorder. Those in need of treatment include those already with the disorder as well as those prone to have the disorder or those in whom the disorder is to be prevented.

An “effective amount” of isolated clusterin or an isolated polypeptide substantially the same as clusterin is an amount needed to seal the occur surface barrier against fluorescein staining.

An “effective amount” may be determined empirically and in a routine manners in relation to the stated purpose.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS9™. Preservatives such as benzylalkonium chloride can also be included.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical foul's, such as dimers or alternatively glycosylated or derivatized forms, or synthetic peptides.

The term “substantially the same” refers to nucleic acid or amino acid sequences having sequence variation that do not materially affect the ability of the amino acid sequence to prevent uptake of clinical fluorescein dye. With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function. A His tag can be added to the end of a protein to aid in purification and tracking in PK/PD assays.

### Pharmaceutical Compositions

One aspect of the present invention is directed to a pharmaceutical composition comprising an isolated clusterin or an isolated polypeptide substantially the same as clusterin. Preferably, the clusterin is secreted clusterin. Preferably, the pharmaceutical composition comprises a carrier, and even more preferably the carrier is a sterile solution.

Human clusterin (CLU) is composed of two disulfide-linked α (34-36 kD) and β (36-39 kD) subunits derived from a single amino acid chain (449 amino acids in human) that becomes glycosylated in the endoplasmic reticulum and Golgi bodies and undergoes intramolecular cysteine bonding and proteolytic cleavage before secretion. The first 22 amino acids comprise the secretory signal sequence. The cleavage site between the α and β chains is between amino acids 227 and 228. Clusterin contains three hydrophobic domains, a long α-helix motif near the amino terminal and at least six N-linked glycosylation sites. It also contains five amphipathic helices which are thought to mediate binding to a variety of normal and denatured proteins and may be important for binding the ocular surface.

The sequence listing of Clusterin Isoform 2 Preproprotein [Homo sapiens] (NCBI Reference Sequence: NP_976084.1) is as follows:

ORIGIN

1 mmktlllfvg Illtwesgqv lgdqtvsdne lqemsnqgsk yvnkeiqnav ngvkqiktli

61 ektneerktl lsnleeakkk kedalnetre setklkelpg vcnetmmalw eeckpclkqt

121 cmkfyarvcr sgsglvgrql eeflnqsspf yfwmngdrid sllendrqqt hmldvmqdhf

181 srassiidel fqdrfftrep qdtyhylpfs 1phrrphfff pksrivrslm pfspyepinf

241 hamfqpflem iheaqqamdi hfhspafqhp ptefiregdd drtvcreirh nstgclrmkd

301 qcdkcreils vdcstnnpsq aklrreldes lqvaerltrk ynellksyqw kmlntsslle

361 qlneqfnwvs rlanitqged qyylrvttva shtsdsdvps gvtevvvklf dsdpitvtvp

421 vevsrknpkf metvaekalq eyrkkhree

In vivo, the human precursor polypeptide chain is cleaved proteolytically to remove the 22 amino acid secretory signal peptide and subsequently between residues 227/228 to generate the alpha and beta chains. These are assembled in an anti-parallel fashion to give a heterodimeric molecule in which the cysteine-rich centers are linked by five disulfide bridges and are flanked by two predicted coiled-coil alpha-helices and three predicted amphipathic alpha-helices.

The clusterin of the present invention can be human clusterin, including secreted clusterin and/or nuclear clusterin, or any subunit, fragment or region of either capable of preventing uptake of clinical fluorescein dye. Acceptable subunits may include human or secreted clusterin without the secretary signal sequence. The term clusterin also encompasses polypeptides with optional non-peptidic components, such as carbohydrate groups or any other non-peptidic substituents that may be added to clusterin by a cell in which the protein is produced, and may vary with the type of cell.

Recombinant human clusterin may be purchased from any number of known sources, expressed in cell lines of mouse and human. It may also be isolated from human serum by known methods. Any subunit, fragment or region may be isolated or synthesized according to known techniques for polypeptide synthesis. Human recombinant clusterin with the His tag added can be expressed in human HEK293 cells or other appropriate cell lines. The production should be done under GMP conditions if the protein is to be used as a human therapeutic.

The pharmaceutical compositions of the present invention may also include polypeptides substantially the same as human clusterin, secreted clusterin, nuclear clusterin or any subunit, fragment or region of either capable of preventing uptake of clinical fluorescein dye. Generally, amino acid sequences are substantially the same if they have a sequence variation that do not materially affect the ability of the protein, subunit, fragment or region to prevent uptake of clinical fluorescein dye. These polypeptides can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art. The polypeptides of the present invention may be made by known techniques for polypeptide synthesis.

The polypeptides of the present invention which occur naturally, or are synthesized according to known methods, are generally “isolated.” Specifically, the polypeptides should be used in the pharmaceutical composition of the present invention in a condition other than their respective native environment, such as apart from blood and animal tissue. In a preferred embodiment, the isolated polypeptide is substantially free of other polypeptides, particularly other polypeptides of animal origin. It is preferred to provide the polypeptides in a highly purified form, i.e. greater than 95% pure, more preferably greater than 99% pure.

The administration of the clusterin phaiiiiaceutical composition is generally topical, with administration of the composition to the surface of the eye in drops).

Compositions and formulations for topical administration can include sterile aqueous solutions that can also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions can be generated from a variety of components that include, but are not limited to, preformed liquids, self-emulsifying solids and self-emulsifying semisolids.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention can also be formulated as suspensions in aqueous, non-aqueous or mixed media. Aqueous suspensions can further contain substances that increase the viscosity of the suspension including, for example, sodium carboxymethylcellulose, sorbitol and/or dextran. The suspension can also contain stabilizers.

The compositions of the present invention can additionally contain other adjunct components conventionally found in pharmaceutical compositions. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosages and desired drug concentrations of pharmaceutical compositions of the present invention may vary depending on the particular use envisioned. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

Dosing is also dependent on severity and responsiveness of the disease state to be treated, with the course of treatment lasting from several days to several months, or until symptomatic relief or a cure is effected or a diminution of the disease state is achieved. Optimum dosages can vary depending on the relative potency of individual polypeptide and should generally be sufficient to prevent uptake of clinical fluorescein dye. Following successful treatment, it can be desirable to have the subject undergo maintenance therapy to prevent the recurrence of the disease state, wherein the polypeptide is administered in maintenance doses.

An especially preferred dosage form is a sterile solution for topical use, such as use as drops. Therapeutic formulations are prepared for storage by mixing the active ingredient having the desired degree of purity with optional physiologically acceptable liquid carrier, and optionally other excipients or stabilizers (Remington's Pharmaceutical Sciences 16th edition, Osol, A. Ed. (1980)), to produce an aqueous solution or suspension. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol.

The solution or suspension formulations should be sterile. This is readily accomplished by filtration through sterile filtration membranes, prior to or following lyophilization and reconstitution. The resulting therapeutic compositions herein generally are placed into a container and the route of administration is in accord topical administration.

### EXAMPLES

### Materials, Methods and Models

### Proteins and Antibodies

HUGO nomenclature is used for genes and their products, unless otherwise indicated. The secreted form of recombinant human CLU (rhCLU) and recombinant mouse CLU (rmCLU), both of which contain a polyhistidine-tag (His6 tag) at the C-terminus, were purchased from R&D Systems (Minneapolis, Minn.). These proteins are expressed in mammalian cells and are fully glycosylated and processed, closely modeling secreted CLU expressed in vivo. Natural secreted plasma CLU (pCLU) purified from human serum was purchased from ProsPec (Ness-Ziona, Israel). Bovine serum albumin (BSA) was purchased from R&D Systems. The cytokine TNFA was purchased from Sigma (St. Louis, Mo.). Anti-CLU (sc-6419) and anti-LGALS3 antibodies (sc-23983) were purchased from Santa Cruz Biotech (Santa Cruz, Caif.). Anti-OCLN (ab168986), anti-ACTB (ab6276), and anti-His6 tag (ab18184) antibodies were purchased from Abcam (Cambridge, Mass.).

### Preclinical Mouse Model

The University of Southern California's Institutional Animal Care and Use Committee approved the research protocol for use of mice in this study. Research was conducted in adherence with the Association for Research in Vision and Ophthalmology (ARVO) Statement for the Use of Animals in Ophthalmic and Visual Research.

Wild type C57Bl/6J female mice purchased from Jackson Labs (Bar Harbor, ME) were used for all experiments unless otherwise stated. Mice were housed in a pathogen-free barrier facility at USC and kept at 25±1° C., relative humidity 60%±10%, with alternating 12 h light/dark cycles. Euthanasia was performed using compressed CO₂ gas, according to the American Veterinary Medical Association Guidelines for the Euthanasia of Animals: 2013 Edition.

Desiccating stress was induced in 6-8 week old mice by the air-draft-plus-scopolamine protocol, as previously described [14]. Briefly, scopolamine hydrobromide (Sigma-Aldrich, St. Louis, Mo.) (0.5 mg/0.2 mL in PBS) was injected subcutaneously in alternating hindquarters, 4 times/day (7 AM, 10 AM, 1 PM, and 4 PM), to inhibit tear secretion. At the same time, mice were exposed to an air draft for 18 hours/day in a room with 80±1° F. and <40% humidity at all times. Standard desiccating stress induction was done for 5 days, otherwise, for the period as indicated.

Delivery of CLU was performed as previously described [8, 14]. Eye drops of CLU or BSA were formulated in PBS vehicle and drops were delivered topically to the unanesthetized mouse eye. The standard treatment protocol was 1 μL/eye, 4 times/day, delivered at the time of scopolamine injection. In some experiments drops were delivered a single time. PBS alone was used as the vehicle control.

Corneal epithelial uptake of clinical fluorescein dye Fluoresoft®-0.35% (Holies Laboratory, Cohasset, Mass.) was assessed quantitatively using fluorometry, as previously described [14]. In some experiments as noted, Alexa-Fluor-dextran (Molecular Probes, Eugene, Oreg.) was substituted.

### Imaging of Fluorescein Uptake at the Ocular Surface

Laser scanning confocal microscopy was used to image the punctate pattern of fluorescein uptake, as described [42]. Mice were euthanized following treatment and whole eyes were extracted. The eyes were immersed in PBS while the optic nerve was detached, following which they were placed anterior side up, on a 0.8% agarose plate (NuSieve® GTG® Agarose, Lonza, Rockland, Me.). Whole mount digital images (512×512 pixels) were captured with a laser-scanning confocal microscope (LSM 5 Pascal, Zeiss, Thornwood, NY) using a 10× objective. Fluorescent images in the central cornea of the samples were captured in Z-section at 1 μm intervals by using identical photomultiplier tube gain settings and processed using Zen 2012 software (Zeiss) and ImageJ64 software (http://imagej.nih.gov/ij/). The individual layers of the corneal epithelium were captured utilizing the Z-stack option. This technique allows for the specimen to be scanned from the surface to the basal layer of the epithelium. The Z-stack can then be projected into a flat image representing fluorescein uptake through all layers of the epithelium. The software can also combine the Z-stack images into a three-dimensional (3-D) configuration, generating a cross section that is perpendicular to the apical plane. In this way, penetration of fluorescein into the apical, sub-apical, and basal layers of the epithelium can be evaluated.

### Imaging of CLU Binding to the Ocular Surface and LGALS3 Affinity Chromatography

CLU binding to the ocular surface was visualized using an indirect immunofluorescent labeling technique and imaged by laser scanning confocal microscopy as described above. Antibody (50 μg) to the His6 tag on rhCLU was labeled with CF™-594 (excitation/emission 593 nm/614 nm) using a CF dye SE protein labeling kit (Biotium, Hayward, Calif.). The final labeled antibody was prepared in PBS at 1.7 mg/mL after removal of unincorporated dye molecules. CLU-CF-594-Ab complex (CLU at ˜110 μg/mL, which is >threshold concentration) was made before instillation to the ocular surface by incubating CLU (2 μL, of 200 μg/mL) and labeled antibody (1.5 μL of 1.7 mg/mL) in the dark for 3 h at room temperature (RT). To each eye, 2 μL of CF-594-Ab alone or complex solution was applied for 15 min before extracting eyes for imaging. As a reference point for CLU binding on the ocular surface, eyes in some experiments were co-treated for 5 min before extraction with a fluorescent lipophilic membrane tracer DiO (1 μL) (3,3′-Dioctadecyloxacarbocyanine Perchlorate, Life Technologies; excitation/emission=484/501 nm), which was dissolved at 1 μg/mL DMSO.

LGALS3 affinity chromatography was performed as previously described [3]. The CLU present in the various collected fractions was quantified by Western blotting.

### Apoptosis Assay and Epithelial Protein Analysis

After 7-day DS with PBS or CLU (1 μg/ml) treatments, eyes were frozen in OCT solution and cross-sectioned at 10 μm thickness. To detect apoptosis, tissue slides were stained for the terminal deoxynucleotidyl transferase dUTP nick end labeling (TUNEL) using the In Situ Cell Death Detection Kit Fluorescein (Sigma-Aldrich) according to the protocol provided by the company with permeabilization for 12 min at 37° C., and the fluorescent images were obtained by confocal microscopy.

Protein preparation from epithelial tissue lysates was described previously [23]. Protein extracts from individual eyes in the same treatment group (7 mice/treatment) were pooled. 20 μg of protein/sample was resolved by denaturing SDS-PAGE (12% gel) for Western blotting.

### Cell Culture Model

Cells of the telomerase-immortalized human corneal limbal epithelial cell (HCLE) line [43] were plated in a 96-well plate and left for 7 days to stratify and differentiate, as previously described [23]. To measure secreted MMP9 produced in response to treatments, cell conditioned media samples were subjected to gelatin zymography and Image J analysis [23].

### Tear CLU Quantification

Mouse basal tears were collected in mice by instillation of 2 μL of PBS containing 0.1% BSA into the conjunctival sac of each eye, which was then collected with a glass capillary tube from the tear meniscus in the lateral canthus as described [44]. Samples were pooled from 2 eyes. Tear volume was measured using phenol red-impregnated cotton threads (Zone-Quick; Oasis, Glendora, Calif.) [45]; results were similar to tear volumes reported previously [46]. CLU was quantified using the Mouse Clusterin Quantikine ELISA kit (R&D Systems), according to the protocol provided by the company, which utilized a standard curve.

### CLU Knockout Mice

In some experiments, CLU knockout mice on the C57Bl/6J background were used. Heterozygous breeders were purchased from Jackson Labs and bred with C57Bl/6J wild type mice to obtain both heterozygotes and homozygotes on the same background. Genotypes of offspring were confirmed by PCR from genomic DNA isolated from tail tips. The PCR primers were previously described [34].

A morphological evaluation was performed on the unstressed ocular surface of CLU knockout mice of both the heterozygous CLU^{+/-} and homozygous CLU^{-/-} genotypes, comparing to wild type C57Bl/6J mice. First, a hand-held 20-diopter indirect lens was used to examine the ocular surface. The ocular surface of eyes from two different mice of the heterozygous CLU^{+/-} genotype was compared to eyes from two different mice of the wild type genotype. Mice were not anesthetized, nor was any topical anesthetic applied to the ocular surface prior to examination. An ophthalmologist and cornea sub-specialist (MH) performed the evaluation. Similarly, the ocular surface of eyes from three different mice of the homozygous CLU^{-/-} knockout genotype was evaluated.

Next, the ocular surface was evaluated by histologic techniques. Briefly, eyes were fixed in 4% formaldehyde and embedded in paraffin. Sections of 6 μm were stained with hematoxylin and eosin or periodic acid-Schiff reagent and photographed with a Nikon Eclipse E400 (Garden City, N.Y.) microscope equipped with a Nikon DXM 1200 digital camera. One eye from each of three different mice was examined from the WT, heterozygous CLU^{+/-} or homozygous CLU^{-/-} genotypes (nine eyes total).

Ocular surface ultrastructure was evaluated by transmission electron microscopy. Briefly, a slit was made at the corneal-scleral margin of the eye, which was then immersed in 2% glutaraldehyde, 2% paraformaldehyde in sodium cacodylate buffer, pH7.4, containing 0.025% (w/v) CaCl2, for 60 min at RT. Anterior segments were separated from the lens and posterior segments and held in fixative overnight before being post-fixed in 1% osmium tetroxide and embedded in EmBed (EMS) resin. Thin sections (70 nm) were post-stained with uranyl acetate and lead citrate, viewed in a JEOL 1200 electron microscope, and photographed with an AMT XR-41 TEM digital camera. One eye from each of three different mice was examined from the WT or homozygous CLU^{-/-} genotypes (six eyes total). An ocular pathologist (GRK) evaluated the images.

### Statistical Analyses

Treatment groups (DS+PBS versus DS+CLU or DS+BSA) were compared to controls (non-stressed (NS) versus DS) on the continuous study variables with generalized linear regression models, using an identity link function. In the regression model, a generalized estimating equation approach was used to explicitly incorporate the correlated outcomes between eyes within one animal [47, 48]; an exchangeable correlation structure was used. The independent sample t-test was used to compare cell culture results between groups. Two-sided P ≤0.05 was considered statistically significant. Analyses were performed using the Statistical Analysis Software (SAS, Version 9.4).

### Example 1

### Topical CLU Protects the Ocular Surface Subjected to Desiccating Stress

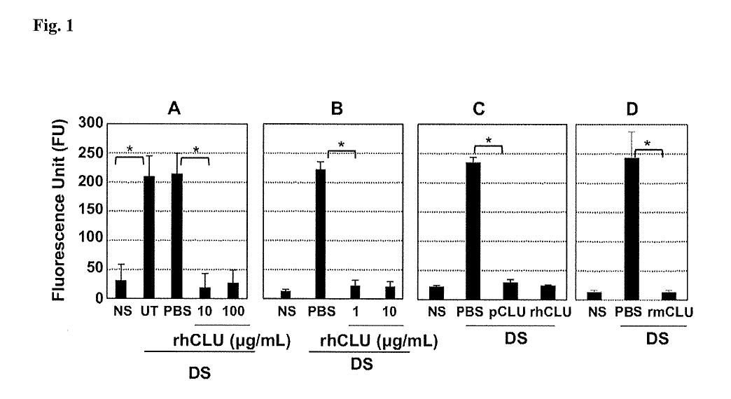

To determine whether supplementation with topical CLU could protect against disruption of the ocular surface barrier subjected to desiccating stress, we applied the 5-day desiccating stress protocol to mice, and also treated topically with recombinant human CLU (rhCLU) formulated in PBS, applied 4 times/day at the same time as scopolamine was administered. After 5 days, barrier integrity was quantified by measuring uptake of fluorescein dye. Results were compared to controls treated with PBS vehicle alone. The stressed but untreated (UT) ocular surface served as the control for PBS treatment and non-stressed (NS) eyes served as the baseline control. Since CLU concentration in human serum was known to be in the range of 100±50 μg/mL [49], we used 10 or 100 μg/mL of rhCLU for our first experiments (FIG. 1A). Dye uptake in stressed eyes treated with PBS alone was ˜8-fold greater than NS counterparts. In contrast, dye uptake in eyes that were stressed, while also being treated with CLU at 10 or 100 μg/mL, was similar to that of NS counterparts, indicating complete protection against barrier disruption. We performed a second set of experiments using a 7-day desiccating stress protocol and rhCLU concentrations of 1 and 10 μg/mL. Again we observed nearly complete protection against barrier disruption as measured by dye uptake at both concentrations (FIG. 1B). We performed a similar experiment using a 5-day desiccating stress protocol, but using human plasma CLU (pCLU) (FIG. 1C) or recombinant mouse CLU (rmCLU) (FIG. 1D) to rule out the possibility that the results might be unique to rhCLU. Treatment with 2 μg/mL of pCLU or rmCLU consistently protected against barrier disruption as measured by fluorescein uptake, to the same extent as rhCLU at 2 μg/mL, and was comparable to NS controls.

### Example 2

### Topical CLU Protects the Ocular Surface in an All-or-None Response

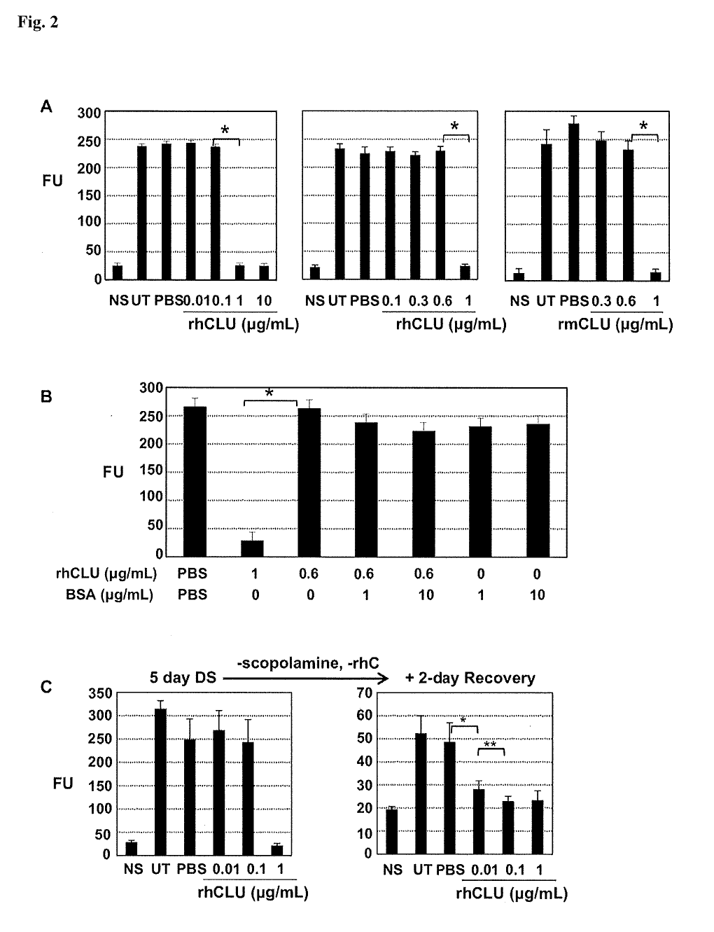

To determine a dose-response for barrier protection by CLU, we next applied the 5-day desiccating stress protocol while simultaneously treating the ocular surface with serial 10-fold dilutions of rhCLU. Similar to results of the experiment shown above (FIG. 1), treatment with 1 μg/mL or 10 μg/mL almost completely protected against fluorescein uptake. In contrast, lower concentrations had essentially no effect, with values similar to UT and PBS-treated groups (FIG. 2A Left). To determine any gradation in activity between 0.1 and 1 μg/mL CLU, we tested CLU concentrations at tight intervals in between these doses (FIG. 2A Middle). We observed a transition in effectiveness between 0.6 μg/mL and 1 μg/mL, essentially an all-or-none response. We also tested rmCLU; the dose transition was at exactly the same place, between 0.6 and 1 μg/mL (FIG. 2A Right). Next, we tested whether BSA, as an in vitro protein stabilizer and as a non-CLU protein also found in serum, could enhance the protective activity of CLU at the low concentration. BSA did not show any significant protective or enhancing effect, alone or with CLU at 0.6 μg/mL, compared with 1 μg/mL of CLU alone (FIG. 2B). Use of Alexa-Fluor-dextran in the fluorescein uptake assay, which is more discriminating because of its much larger molecular size [14], gave identical results (data not shown).

To determine whether a dose response effect could be observed at CLU concentrations below the threshold level exhibited by the sharp transition, we changed our experimental conditions. As before, we applied the 5-day desiccating stress protocol while simultaneously treating the ocular surface with rhCLU, but then on day 6 we stopped CLU treatment and discontinued scopolamine injections, but maintained a milder stress by continuing the air draft, elevated temperature and reduced humidity. We then waited an additional two days, following which time we assayed barrier integrity by fluorescein dye uptake (FIG. 2C). Disruption of the ocular surface barrier after the 2-day moderate desiccating stress was considerably less than observed when the dye uptake assay was done directly following the 5-day desiccating stress protocol. Interestingly, in this setting, we found that the prior delivery of 0.1 μg/mL CLU, 4 times/day was as effective as 1 μg/mL. Again the result was primarily all-or-none, although we observed a small graded effect between 0.01-0.1 μg/mL, which may reflect the transition between desiccating stress conditions.

These results indicate that topical CLU protects the ocular surface barrier against disruption by desiccating stress in an all-or-none manner at a very precise threshold dose range that is highly reproducible.

### Example 3

### Topical CLU Ameliorates Pre-existing Ocular Surface Barrier Disruption due to Desiccating Stress

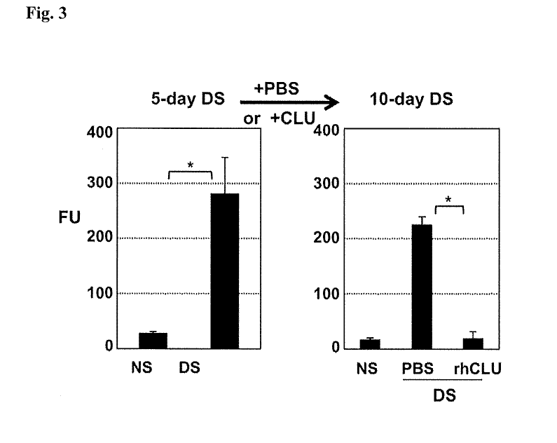

Having clearly demonstrated the preventive effect of CLU in protecting the ocular surface against desiccating stress, we next assessed the potential of CLU to ameliorate pre-existing ocular surface disruption. Representative results are shown in FIG. 3. In this experiment, we applied the 5-day desiccating stress protocol, and then treated topically with rhCLU at 2 μg/mL (4 times/day) for another 5 days while maintaining the same desiccating stress protocol. Following this, barrier integrity was assayed. The PBS control showed a high level of dye uptake, ˜12× greater than NS controls, but the barrier was essentially intact in CLU treated mice, similar to NS controls.

### Example 4

### Topical CLU Directly Seals the Ocular Surface Barrier Against Disruption due to Desiccating Stress

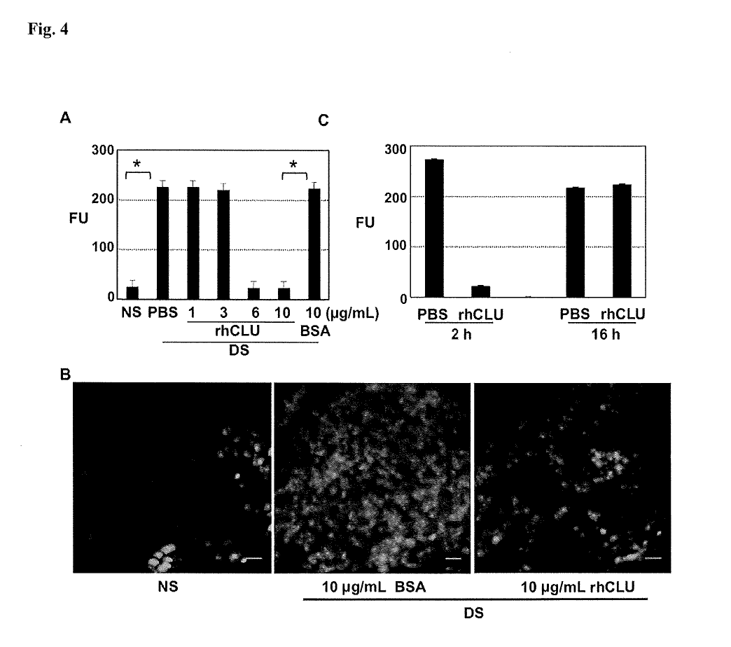

The amelioration results outlined above (FIG. 3) suggested that one of the mechanisms of CLU action might be simply to seal areas of barrier damage so that dye can no longer penetrate. To test this idea, we applied the 5-day desiccating stress protocol, and then treated with CLU, but this time assayed for dye uptake within 15 minutes of treatment, giving the ocular surface no time to recover from the stress (FIG. 4A). An all-or-none response was observed once again, but the transition point was higher than when CLU was applied 4 times/day. Thus CLU at 6 μg/mL, applied one time, was completely effective in preventing dye uptake, while 3 μg/mL was completely ineffective. Laser scanning confocal microscopy was used to visualize punctate staining and its amelioration (FIG. 4B). Eyes of mice subjected to desiccating stress and treated with BSA control showed many punctate spots of the size and shape of cells, similar to UT eyes, while desiccating stress eyes treated with CLU at 10 μg/mL showed far fewer spots, similar to the non-stressed control. In a second set of experiments we sought to determine how long the sealing effect would last. In a time course experiment, the sealing effect was maintained for 2 hours, but was lost by 16 hours (FIG. 4C).

### Example 5

### Topical CLU Binds Selectively to the Ocular Surface Subjected to Desiccating Stress, and to LGALS3 in vitro

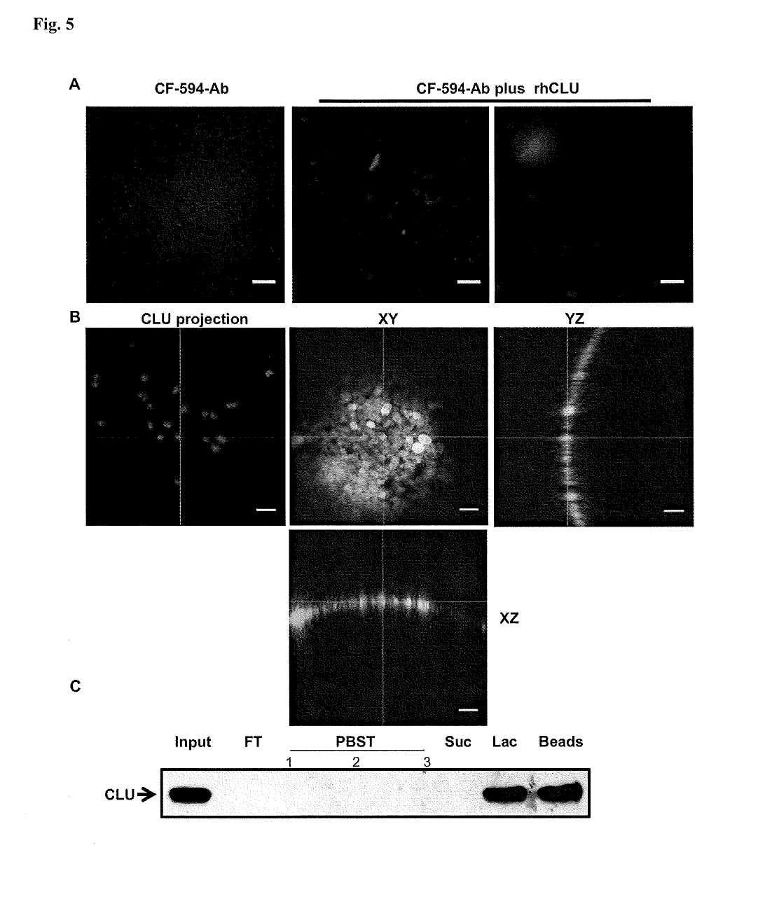

To visualize CLU binding to the ocular surface, we used the technique of direct immunostaining with an antibody conjugated to CF-594 dye. To differentiate topically applied rhCLU from endogenous CLU, we took advantage of the C-terminal His tag incorporated into the rhCLU molecule. Representative results are shown in FIG. 5A. Eyes subjected to desiccating stress, then treated with CF-594 dye conjugated anti-His antibody alone, showed some diffuse fluorescence over the ocular surface subjected to desiccating stress. However, when the ocular surface of these mice was treated with rhCLU, substantial punctate binding of dye-conjugated antibody to the ocular surface subjected to desiccating stress was observed, indicating the location of direct CLU binding. In contrast, the NS eye showed far less binding. In a second set of experiments, the fluorescent lipophilic membrane tracer DiO was used to delineate individual cells. Representative results are shown in FIG. 5B. This showed that the CLU “spots” were approximately the size of cells. In some cases, the CLU spots (red) filled the entire area of individual cells marked by the dyed membrane (green), overlapping completely (yellow color). In other cases, CLU spots were clearly separate.

Next we considered what kinds of ocular surface molecules might bind CLU. LGALS3, a key component of the ocular surface barrier, is a member of the galectin class of beta-galactoside-binding proteins. What is known about the glycosyl moiety of CLU is consistent with LGALS3 binding [25, 27]. CLU applied to an LGALS3-sepharose affinity column bound to the beads and was not eluted 0.1 M sucrose, a disaccharide that does not compete with LGALS3 sugar binding, but was mostly eluted with a competitive inhibitor of LGALS3 sugar binding, 0.1 M beta-lactose (FIG. 5C). This suggests that CLU binding to LGALS3 is specific for the beta-galactoside-binding function.

### Example 6

### Topical CLU is Cytoprotective and Proteostatic

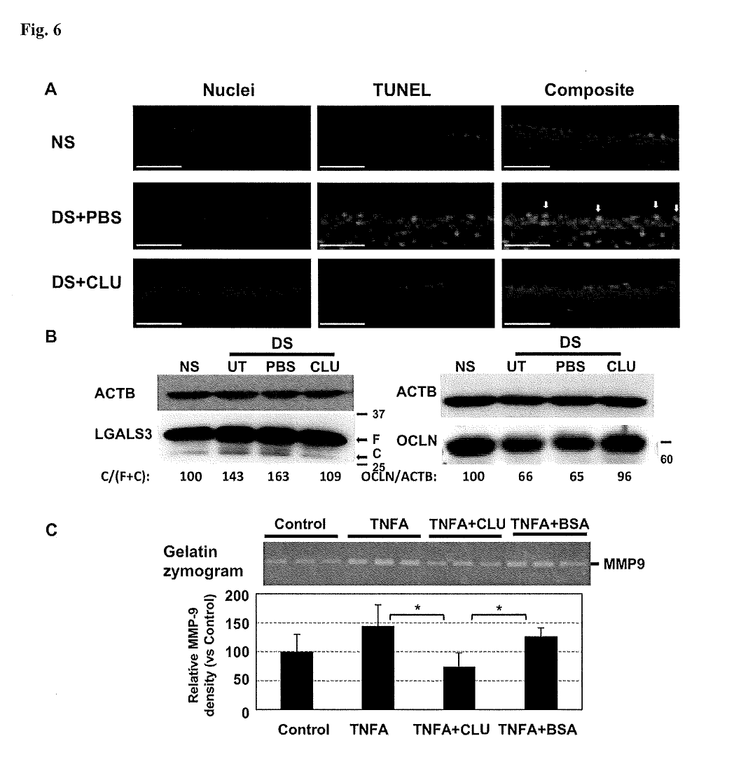

Having demonstrated the capacity of CLU to protect the ocular surface barrier against functional disruption due to desiccating stress, we next tested its capacity to protect the cells and proteins of the barrier against physical damage. First we investigated the cytoprotective activity of topical CLU. Representative results are shown in FIG. 6A. Only a few cells at the ocular surface of non-stressed (NS) eyes were positively stained in the TUNEL assay, a measure of DNA damage characteristic of apoptotic cells. In the PBS-treated DS eye, staining of epithelial cells and stroma cells was strikingly increased, consistent with previous observations [11-13, 50]. However, when the ocular surface was treated topically with CLU at the same time as it was subjected to desiccating stress, the level of TUNEL staining remained the same as in non-stressed eyes.

We next investigated protection of ocular surface barrier proteins against desiccating stress. Representative results are shown in FIG. 6B. Corneal epithelial lysates were isolated from the eyes of mice maintained under ambient conditions (NS), mice subjected to desiccating stress but otherwise untreated (UT), and mice subjected to desiccating stress while also being treated with rhCLU or the PBS control. We found an increase in a truncated form of LGALS3 after desiccating stress, which suggested proteolysis. Importantly, LGALS3 was protected from truncation in the corneal epithelium of mice treated with topical CLU in PBS, but not in mice treated with PBS alone. Similarly, the amount of the tight junction protein OCLN was reduced in the corneal epithelium of eyes subjected to desiccating stress, but was restored in mice treated with topical CLU in PBS, but not when treated with PBS alone. It should be noted that the area of ocular surface balTier damage is expected to be only a small percentage of the total based on the pattern of punctate fluorescein staining. These findings provide evidence that CLU protects the protein structure of both the transcellular and paracellular barriers at the mouse ocular surface subjected to desiccating stress.

We also examined the effect of CLU on MMP9 expression using a corneal cell culture model, as shown in FIG. 6C. Treatment of cells with rhCLU significantly reduced (by ˜50%) the stimulatory effects of TNFA on MMP9 expression, but BSA had no effect. These results provide a second possible mechanism for ocular surface barrier protection against proteolysis.

### Example 7

### Causal Association between CLU Concentration in Tears and Ocular Surface Barrier Vulnerability

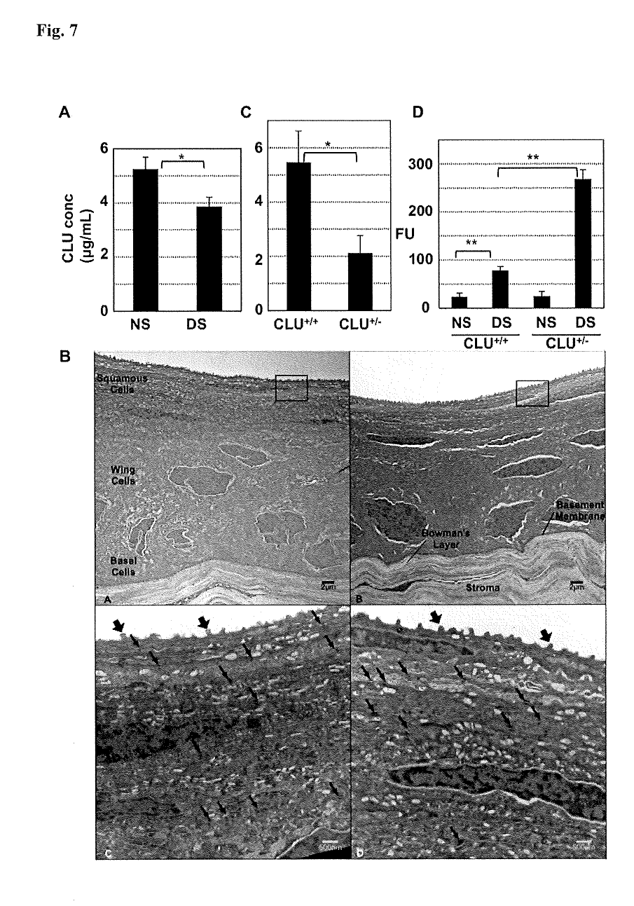

The concentration of endogenous CLU in mouse tears was measured using an ELISA. Representative results are shown in FIG. 7A. In this experiment, the mean CLU concentration in tears from mice kept at ambient conditions was 5.2±0.4 μg/mL. This was reduced to 3.7±0.3 μg/mL in tears from mice subjected to the 5-day desiccating stress protocol, an ˜30% reduction, similar to what was previously observed in the ocular surface epithelium using this mouse model [23].

CLU knockout mice could be useful for examining the causal relationship between endogenous CLU concentration in tears and ocular surface barrier vulnerability to desiccating stress if the ocular surface is normal under ambient conditions. On gross inspection, eyes of both heterozygous CLU^{+/-} and homozygous CLU^{+/-} knockout mice on the C57BL/6J background appeared anatomically normal. We examined the ocular surface of both of these knockout genotypes more closely using a hand-held 20-diopter indirect lens, and compared to wild type C57BL6/J mice. In all three genotypes, the tear film appeared of similar thickness and the ocular surface appeared smooth and unaffected, with no inflammatory infiltrates apparent. Histological analysis of cross-sections, revealed no differences among genotypes, and periodic acid-Schiff histochemistry revealed similar goblet cell numbers in all genotypes (data not shown).

Ocular surface epithelia examined by transmission electron microscopy revealed no differences between wild type C57Bl/6J and homozygous CLU^{-/-} knockout mice. Representative images are shown in FIG. 7B. There was no evidence of squamous metaplasia in the corneal or conjunctival epithelia. Microplicae at the apical cell surface appeared similar in contour and density. Junctional complexes between cells were of similar appearance and numbers. Thus the ocular surface of homozygous CLU^{-/-} knockout mice maintained under ambient conditions appears to be entirely normal, i.e., the same as wild type counterparts.

Next we compared tear CLU concentration in WT and heterozygous CLU^{+/-} mice maintained at ambient conditions. Representative results are shown in FIG. 7C. The mean tear CLU concentration in this group of WT mice was 5.511.2 μg/mL, while the mean concentration in heterozygous CLU^{+/-} mice was 2.210.6 μg/mL. This is an ˜50% difference and indicates that the CLU concentration in tears is roughly proportional to the number of gene copies. Significantly, the reduced level of CLU in tears of heterozygous CLU^{+/-} KO mice was less than the level of CLU in tears of WT eyes subjected to desiccating stress. Thus the heterozygous CLU^{+/-} KO genotype can be used to determine whether reduced CLU levels in tears alone results in vulnerability to desiccating stress.

Finally, barrier sensitivity was evaluated in WT and heterozygous CLU^{+/-} KO mice. To facilitate the detection of differences, the mild desiccating stress protocol was used. Thus mice were exposed to air draft at elevated temperature and reduced humidity, but scopolamine injections were omitted (as in FIG. 2C). This protocol was continued for 4-weeks, after which time, ocular surface barrier integrity was assayed. Representative results are shown in FIG. 7D. Fluorescein uptake in WT eyes was only about 3× higher than NS controls. In contrast, fluorescein uptake in heterozygous CLU^{+/-} KO mice was approximately 10× higher than NS controls. These results demonstrate that reduced CLU in the tears correlates with increased vulnerability of the ocular surface barrier to desiccating stress.

CLU is a homeostatic protein, prominently expressed at fluid-tissue interfaces throughout the body including the ocular surface. The present invention demonstrates that CLU prevents and ameliorates ocular surface barrier disruption due to desiccating stress by a remarkable sealing mechanism dependent on attainment of a critical concentration in the tears. When tear CLU drops below the critical threshold, the ocular surface barrier becomes vulnerable to disruption. Sealing by CLU involves selective binding to the stressed ocular surface. Positioned in this way, CLU not only physically seals the ocular surface barrier, but it also protects the barrier cells and prevents further damage to barrier structure. These findings provide an answer to the long mystery of CLU's physiological role at the ocular surface and also identify a fundamentally new mechanism for ocular surface protection.

### Ocular Surface Sealing

Since the ocular surface barrier of the homozygous CLU^{-/-} KO mouse is intact under ambient conditions, it seems unlikely that CLU is a structural component of the nounal barrier, but rather that it serves a protective and surveillance role. This fits with previous reports that CLU knockout mice display a phenotype only when systems are perturbed by application of inflammatory disease models [30, 33, 34]. The selectivity of topical CLU binding for the ocular surface subjected to desiccating stress suggests that CLU seals by binding to areas of barrier disruption. This remains conjectural at this point, as we have not directly demonstrated co-localization with spots of fluorescein uptake, however the punctate character observed for binding of topical CLU at both the normal ocular surface and the ocular surface subjected to desiccating stress is consistent with this idea. Thus we propose that CLU might also act as a “spot weld” at the ocular surface, sealing damage to the barriers where needed.

A previous study suggested that CLU interacts with a lectin-type receptor on liver cells [51] and here we demonstrate CLU interaction with the galectin LGALS3. Galectins are a family of lectin proteins defined by binding specificity for beta-galactoside containing glycans. The main family member at the human ocular surface is LGALS3 (galectin-3) [3, 52, 53]. All galectins have a C-terminal carbohydrate recognition domain, but LGALS3 is unique in also possessing an N-terminal extension with a repeating motif which enables multimer formation [54]. This gives it the capacity to form networks that bridge membrane-associated mucin ectodomains, to organize the ocular surface barrier. MMPs (and likely other proteinases) specifically cleave the multimerization domain from the body of LGALS3, reducing self-association [55-57]. LGALS3 cleavage products are found at the ocular surface and in tears of dry eye patients [58], and we provide evidence here that LGALS3 is cleaved at the mouse ocular surface subjected to desiccating stress. This suggests the possibility that LGALS3 cleavage frees it for interaction with CLU.

CLU sealing may also occur via direct interaction with the plasma membrane of damaged cells. CLU and related apolipoproteins can insert directly into the plasma membrane of cells in the wall of blood vessels [59-61]. This function appears to be due to the special structural features of CLU, in particular the helical amphipathic domains, which confer the properties of a proteinaceous detergent [26]. N-glycosylation sites are located around the disulfide bonds of CLU and may form a scaffold region in clusterin with negatively charged carbohydrates localized to this scaffold. The arms containing the amphipathic helices may extend outward from the scaffold. In this model, CLU resembles a lipid, with the charged head-group being the carbohydrate-covered scaffold of CLU and the hydrophobic tail being the arms. Sealing by CLU may thus be related to the phenomenon of lipid surfactant-mediated “sealing” of plasma membranes damaged by electroporation or other insults, which prevents leakage of fluorescein from preloaded cells [62, 63]. Importantly, insertion of CLU into the vascular wall [59-61] and surfactant-mediated sealing [62, 63] are both cytoprotective. Recently, CLU association with intracellular membranes was also shown to be cytoprotective [64, 65]. The mechanisms of sealing against fluorescein uptake will be very important to define.

### Critical All-or-None Threshold

The observation of a critical threshold for all-or-none sealing by topical and endogenous CLU is also quite unexpected. Previous mass spectrometric analyses have indicated that CLU protein is present in human tears [39-41], however the concentration has never been measured in humans or any other species. Here we determine that the concentration of CLU in the tears of mice maintained under ambient conditions is between 5-6 μg/mL. CLU concentration was reduced by ˜30% (from 5.2 μg/mL to 3.6 μg/mL) in the tears of mice subjected to the 5-day desiccating stress protocol, similar to the percent reduction previously reported in the ocular surface epithelia in this mouse model [23]. Heterozygous CLU^{30 /+} KO mice were found to have about half the tear CLU concentration of wild type mice, as would be predicted by the genetic deficiency. This reduction in concentration (to 2.5 μg/mL) results in increased vulnerability to desiccating stress. Adding CLU by topical application corrects this, resealing the barrier.

Our results suggest that the normal concentration of endogenous CLU in tears is above the critical threshold, thus ensuring that the ocular surface barrier remains sealed when subjected to stress. Desiccating stress reduces tear CLU below the threshold, thus making the ocular surface barrier vulnerable to disruption. This means the critical threshold must be somewhere between 5-6 μg/mL and 3.6 μg/mL. Perhaps significantly, this fits within the range of the all-or-none threshold for sealing by a single topical application of CLU (3-6 μg/mL). We envision that topical CLU applied as a 1 μL drop, which is ˜30-fold larger than the tear volume [46], would dilute the CLU already present in the tears. Thus, tear CLU contribution to total CLU would seem to be negligible, and the topical threshold for sealing constant, regardless of tear concentration. Enigmatically, a much lower critical threshold is observed when CLU is applied multiple times a day. Perhaps this means we are, in fact, supplementing tear CLU with topical CLU. On the other hand, the mechanism could be much more complex. For example, as noted in the Introduction, CLU has anti-inflammatory properties and thus topical CLU treatment might stimulate a recovery of CLU tear levels over time. The all-or-none sealing of the ocular surface barrier disrupted by desiccating stress occurs when the concentration of CLU molecules is high enough to compete effectively with mucins for binding to LGALS3 molecules.

All-or-none responses are seen in many biological processes [66-68] and often involve the assembly of multimeric complexes at a critical concentration [69]. CLU can exist in monomeric or multimeric forms [70, 71] and is found in large complexes in numerous diseases [72-74]. Thus one possible mechanism for the critical threshold effect is that CLU must co-assemble with LGALS3 (and possibly other molecules) into a multimeric complex before it can seal the balTier. Cleavage of LGALS3 alters the carbohydrate binding domain structure of LGALS3 so that it binds more tightly to glycoconjugates [57], and we show here that LGALS3 binds in a lactose dependent manner to CLU. Significantly, surfactant-mediated sealing of cells occurs only when the surfactant molecules reach a critical concentration in solution, enabling micelle formation.

### Cytoprotection and Proteostasis

The inventors believe that this is the first time CLU has been demonstrated to be anti-apoptotic at the ocular surface subjected to desiccating stress, however this CLU activity has been well studied in connection with resistance to chemotherapeutics in cancer [28, 29]. Endogenously secreted CLU is re-internalized within the cell by binding to cell surface receptors of the low-density lipoprotein family such as LRP2 (megalin) [75], LPR8, or VLDLR [76], followed by endocytosis. Binding of CLU to LRP2 induces activation of AKT, which phosphorylates Bad [76]. In addition, internalized CLU binds Ku70/Bax complexes, preventing Bax activation [77], and also stabilizes NF-kappaB and IkappaBalpha[78]. Through each of these pathways, internalized CLU increases cell survival and in this way, topical CLU could prevent cells at the ocular surface from entering the apoptotic pathway when subjected to desiccating stress. We must also consider the possibility that CLU's cytoprotective effect is indirect, a result of its well-known anti-inflammatory activity [30].

An additional means for protecting against apoptosis is suggested by our findings on ocular surface sealing by CLU. As discussed above, mechanisms whereby cells at the ocular surface take up water-soluble dyes are poorly understood. A recent study showed that fluorescein uptake occurs selectively in cultured corneal epithelial cells undergoing apoptosis in response to stress (as opposed to dead cells), suggesting an active transport process [79]. A caveat is that the cultures used in this study were non-confluent, meaning that the tight junction-regulated paracellular barrier would not be fully formed. In addition, the cultures were not stratified, meaning that they would not have expressed cell-associated mucins needed to form the transcellular barrier [23]. Nevertheless, the results suggest the intriguing idea that the immediate sealing of the ocular surface upon topical application of CLU, and the capacity of topical CLU to protect cells from undergoing apoptosis, could be causally linked.

Bound at the ocular surface via LGALS3 or other molecules, CLU would be aptly positioned, not only to seal the ocular surface barrier, but also to prevent its further structural damage. The proteostatic effects of CLU as an extracellular molecule chaperone have been well documented [31, 32]. More recently, we showed that CLU is also a potent inhibitor of MMP9 and other MMPs and protects the paracellular barrier against proteolysis by MMP9 in vitro. In this study, we provide the first evidence that CLU maintains proteostasis at both the transcellular and the paracellular barriers at the ocular surface subjected to desiccating stress in vivo. In addition, using a corneal epithelial cell culture model, we show that CLU reduces MMP9 expression stimulated by the inflammatory cytokine TNFa, providing a second way that CLU might be proteostatic. It should be noted that there are two previously published articles presenting data that CLU stimulates MMP9 expression in cell culture models: leukocytes [80] and tumor cells [81]. We do not consider these results to be conflicting with our own, as CLU activities are often seen to be enigmatic, and may be context-dependent [24]. It is well known that MMP expression can be induced by providing aggregated molecules to stimulate phagocytosis [82], thus the aggregation or multimerization status of CLU may make a difference in its effects on MMP expression.

### CLU as a Biotherapeutic for Dry Eye

Our results demonstrate that topical CLU is remarkably protective of the ocular surface in mice, and can completely reverse the primary sign of dry eye, fluorescein staining. The bioavailability of drugs topically applied to the ocular surface is on the order of 5% or less, due to tear washout effects and the permeability barrier [83, 84], however we show that CLU binds to the ocular surface and remains effective for many hours. These findings, combined with the observed cytoprotective and proteostatic effects of CLU, and considered in context of CLU's well-characterized anti-inflammatory properties, present a compelling evidence for CLU as a biological therapeutic for dry eye. As a natural homeostatic protein, CLU would be safe and well tolerated, making it an ideal drug. While non-eukaryotic expression systems have been problematic, hrCLU expressed in mammalian cells is full glycosylated, proteolytically processed, and fully functional as a molecular chaperone [85]. Here we show the hrCLU expressed in mammalian cells is functionally indistinguishable from CLU purified from human plasma in protection and sealing of the ocular surface against desiccating stress.

Cyclosporine A (Restasis®, Allergan) is currently the only FDA approved medication for dry eye [86]. The current standard for FDA approval is two studies showing a statistically significant superiority of the drug to its vehicle in relieving both a sign, e.g. fluorescein uptake, and a symptom, e.g., irritation, dryness, gritty feeling and burning [87, 88]. Consistent amelioration of fluorescein uptake has been a difficult criterion for investigational drugs to meet [86-88]. If the all-or-none effect of CLU treatment in mice holds in humans, the “all” part would be an important advantage.

### REFERENCES

All references cited herein, including those below and including but not limited to all patents, patent applications, and non-patent literature referenced below or in other portions of the specification, are hereby incorporated by reference herein in their entirety.

1. Gipson I K. The ocular surface: the challenge to enable and protect vision: the Friedenwald lecture. Investigative ophthalmology & visual science. 2007; 48(10):4390; 1-8. Epub 2007 Sep. 28. doi: 10.1167/iovs.07-0770. PubMed PMID: 17898256; PubMed Central PMCID: PMC2886589.

2. Blalock T D, Spurr-Michaud S J, Tisdale A S, Heimer S R, Gilmore M S, Ramesh V, et al. Functions of MUC16 in corneal epithelial cells. Investigative ophthalmology & visual science. 2007; 48(10):4509-18. Epub 2007 Sep. 28. doi: 10.1167/iovs.07-0430. PubMed PMID: 17898272.

3. Argueso P, Guzman-Aranguez A, Mantelli F, Cao Z, Ricciuto J, Panjwani N. Association of cell surface mucins with galectin-3 contributes to the ocular surface epithelial barrier. The Journal of biological chemistry. 2009; 284(34):23037-45. Epub 2009 Jun. 27. doi: 10.1074/jbc.M109.033332. PubMed PMID: 19556244; PubMed Central PMCID: PMC2755710.

4. Mantelli F, Massaro-Giordano M, Macchi I, Lambiase A, Bonini S. The cellular mechanisms of dry eye: from pathogenesis to treatment. Journal of cellular physiology. 2013; 228(12):2253-6. Epub 2013 May 23. doi: 10.1002/jcp.24398. PubMed PMID: 23696296.

5. Gipson I K, Spurr-Michaud S, Tisdale A, Menon B B. Comparison of the Transmembrane Mucins MUC1 and MUC16 in Epithelial Barrier Function. PloS one. 2014; 9(6):e100393. Epub 2014 Jun. 27. doi: 10.1371/journal.pone.0100393. PubMed PMID: 24968021; PubMed Central PMCID: PMC4072602.

6. TFOS. Report of the International Dry Eye Workshop (DEWS). The ocular surface. 2007; 5:65-204.

7. Schaumberg D A, Sullivan D A, Dana M R. Epidemiology of dry eye syndrome. Adv Exp Med Biol. 2002; 506(Pt B):989-98. PubMed PMID: 12614022.

8. De Paiva C S, Corrales R M, Villarreal A L, Farley W J, Li DQ, Stern M E, et al. Corticosteroid and doxycycline suppress MMP-9 and inflammatory cytokine expression, MAPK activation in the corneal epithelium in experimental dry eye. Experimental eye research. 2006; 83(3):526-35. Epub 2006 Apr. 29. doi: 10.1016/j.exer.2006.02.004. PubMed PMID: 16643899.

9. Li D Q, Luo L, Chen Z, Kim H S, Song X J, Pflugfelder S C. JNK and ERK MAP kinases mediate induction of IL-lbeta, TNF-alpha and IL-8 following hyperosmolar stress in human limbal epithelial cells. Experimental eye research. 2006; 82(4):588-96. Epub 2005 Oct. 06. doi: 10.1016/j.exer.2005.08.019. PubMed PMID: 16202406; PubMed Central PMCID: PMC2198933.

10. Pflugfelder S C, Jones D, Ji Z, Afonso A, Monroy D. Altered cytokine balance in the tear fluid and conjunctiva of patients with Sjogren's syndrome keratoconjunctivitis sicca. Current eye research. 1999; 19(3):201-11. Epub 1999 Sep. 17. PubMed PMID: 10487957.

11. Luo L, Li D Q, Pflugfelder S C. Hyperosmolarity-induced apoptosis in human corneal epithelial cells is mediated by cytochrome c and MAPK pathways. Cornea. 2007; 26(4):452-60. Epub 2007 Apr. 26. doi: 10.1097/ICO.0b013e318030d259. PubMed PMID: 17457195.

12. Chen W, Zhang X, Liu M, Zhang J, Ye Y, Lin Y, et al. Trehalose protects against ocular surface disorders in experimental murine dry eye through suppression of apoptosis. Experimental eye research. 2009; 89(3):311-8. Epub 2009 Apr. 7. doi: 10.1016/j.exer.2009.03.015. PubMed PMID: 19345212.

13. Clouzeau C, Godefroy D, Riancho L, Rostene W, Baudouin C, Brignole-Baudouin F. Hyperosmolarity potentiates toxic effects of benzalkonium chloride on conjunctival epithelial cells in vitro. Molecular vision. 2012; 18:851-63. Epub 2012 Apr. 25. PubMed PMID: 22529703; PubMed Central PMCID: PMC3332130.

14. Pflugfelder S C, Farley W, Luo L, Chen L Z, de Paiva C S, Olmos L C, et al. Matrix metalloproteinase-9 knockout confers resistance to corneal epithelial barrier disruption in experimental dry eye. The American journal of pathology. 2005; 166(1):61-71. Epub 2005 Jan. 6. doi: 10.1016/S0002-9440(10)62232-8. PubMed PMID: 15632000; PubMed Central PMCID: PMC1602302.

15. Li D Q, Pflugfelder SC. Matrix metalloproteinases in corneal inflammation. The ocular surface. 2005; 3(4 Suppl):5198-202. Epub 2007 Jan. 12. PubMed PMID: 17216119.

16. Foulks G N, Pflugfelder S C. New testing options for diagnosing and grading dry eye disease. American journal of ophthalmology. 2014; 157(6):1122-9. Epub 2014 Mar. 19. doi: 10.1016/j.ajo.2014.03.002. PubMed PMID: 24631478; PubMed Central PMCID: PMC4062650.

17. Abelson M B, Ingerman A. The Dye-namics of Dry-Eye Diagnosis. Review of Ophthalmology on-line. 2005. Epub 15 Nov 2005.

18. Bron A J, Argueso P, Irkec M, Bright F V. Clinical staining of the ocular surface: mechanisms and interpretations. Progress in retinal and eye research. 2015; 44:36-61. doi: 10.1016/j .preteyeres.2014.10.001. PubMed PMID: 25461622.

19. Wiechmann A F, Ceresa B P, Howard E W. Diurnal variation of tight junction integrity associates inversely with matrix metalloproteinase expression in Xenopus laevis corneal epithelium: implications for circadian regulation of homeostatic surface cell desquamation. PloS one. 2014; 9(11):e113810. doi: 10.1371/journal.pone.0113810. PubMed PMID: 25412440; PubMed Central PMCID: PMC4239109.

20. Luo L, Li D Q, Doshi A, Farley W, Corrales R M, Pflugfelder SC. Experimental dry eye stimulates production of inflammatory cytokines and MMP-9 and activates MAPK signaling pathways on the ocular surface. Investigative ophthalmology & visual science. 2004; 45(12):4293-301. Epub 2004 Nov. 24. doi: 10.1167/iovs.03-1145. PubMed PMID: 15557435.

21. Mokhtarzadeh M, Casey R, Glasgow B J. Fluorescein punctate staining traced to superficial corneal epithelial cells by impression cytology and confocal microscopy. Investigative ophthalmology & visual science. 2011; 52(5):2127-35. Epub 2011 Jan. 8. doi: 10.1167/iovs.10-6489. PubMed PMID: 21212176; PubMed Central PMCID: PMC3080172.

22. Chotikavanich S, de Paiva C S, Li de Q, Chen J J, Bian F, Farley W J, et al. Production and activity of matrix metalloproteinase-9 on the ocular surface increase in dysfunctional tear syndrome. Investigative ophthalmology & visual science. 2009; 50(7):3203-9. Epub 2009 Mar. 4. doi: 10.1167/iovs.08-2476. PubMed PMID: 19255163; PubMed Central PMCID: PMC3594995.

23. Jeong S, Ledee D R, Gordon G M, Itakura T, Patel N, Martin A, et al. Interaction of clusterin and matrix metalloproteinase-9 and its implication for epithelial homeostasis and inflammation. The American journal of pathology. 2012; 180(5):2028-39. Epub 2012 Mar. 24. doi: 10.1016/j.ajpath.2012.01.025. PubMed PMID: 22440257; PubMed Central PMCID: PMC3349834.

24. Jones S E, Jomary C. Clusterin. The international journal of biochemistry & cell biology. 2002; 34(5):427-31. Epub 2002 Mar. 22. PubMed PMID: 11906815.

25. Kapron J T, Hilliard G M, Lakins J N, Tenniswood M P, West K A, Can S A, et al. Identification and characterization of glycosylation sites in human serum clusterin. Protein science : a publication of the Protein Society. 1997; 6(10):2120-33. Epub 1997 Oct, 23. doi: 10.1002/pro.5560061007. PubMed PMID: 9336835; PubMed Central PMCID: PMC2143570.

26. Bailey R W, Dunker A K, Brown C J, Garner E C, Griswold M D. Clusterin, a binding protein with a molten globule-like region. Biochemistry. 2001; 40(39):11828-40. Epub 2001 Sep. 26. PubMed PMID: 11570883.

27. de Silva H V, Stuart W D, Park Y B, Mao S J, Gil C M, Wetterau J R, et al. Purification and characterization of apolipoprotein J. The Journal of biological chemistry. 1990; 265(24):14292-7. Epub 1990 Aug. 25. PubMed PMID: 2387851.

28. Toren P J, Gleave M E. Evolving landscape and novel treatments in metastatic castrate-resistant prostate cancer. Asian journal of andrology. 2013; 15(3):342-9. Epub 2013 Apr. 16. doi: 10.1038/aja.2013.38. PubMed PMID: 23584378; PubMed Central PMCID: PMC3739642.

29. Matsumoto H, Yamamoto Y, Shiota M, Kuruma H, Beraldi E, Matsuyama H, et al. Cotargeting Androgen Receptor and Clusterin Delays Castrate-Resistant Prostate Cancer Progression by Inhibiting Adaptive Stress Response and AR Stability. Cancer Res. 2013; 73(16):5206-17. doi: 10.1158/0008-5472.CAN-13-0359. PubMed PMID: 23786771.

30. Savkovic V, Gantzer H, Reiser U, Selig L, Gaiser S, Sack U, et al. Clusterin is protective in pancreatitis through anti-apoptotic and anti-inflammatory properties. Biochemical and biophysical research communications. 2007; 356(2):431-7. Epub 2007 Mar. 16. doi: 10.1016/j.bbrc.2007.02.148. PubMed PMID: 17359935.

31. Humphreys D T, Carver J A, Easterbrook-Smith S B, Wilson M R. Clusterin has chaperone-like activity similar to that of small heat shock proteins. The Journal of biological chemistry. 1999; 274(11):6875-81. Epub 1999 Mar. 6. PubMed PMID: 10066740.

32. Wilson M R, Easterbrook-Smith S B. Clusterin is a secreted mammalian chaperone. Trends in biochemical sciences. 2000; 25(3):95-8. Epub 2000 Mar. 1. PubMed PMID: 10694874.

33. Rosenberg M E, Girton R, Finkel D, Chmielewski D, Barrie A, 3rd, Witte DP, et al. Apolipoprotein J/clusterin prevents a progressive glomerulopathy of aging. Molecular and cellular biology. 2002; 22(6):1893-902. Epub 2002 Feb. 28. PubMed PMID: 11865066; PubMed Central PMCID: PMC135592.

34. McLaughlin L, Zhu G, Mistry M, Ley-Ebert C, Stuart W D, Florio C J, et al. Apolipoprotein J/clusterin limits the severity of murine autoimmune myocarditis. The Journal of clinical investigation. 2000; 106(9):1105-13. Epub 2000 Nov. 9. doi: 10.1172/JCI9037. PubMed PMID: 11067863; PubMed Central PMCID: PMC301413.

35. Aronow B J, Lund S D, Brown T L, Harmony J A, Witte D P. Apolipoprotein J expression at fluid-tissue interfaces: potential role in barrier cytoprotection. Proceedings of the National Academy of Sciences of the United States of America. 1993; 90(2):725-9. Epub 1993 Jan. 15. PubMed PMID: 8421712; PubMed Central PMCID: PMC45738.

36. Trougakos I P, Gonos E S. Clusterin/apolipoprotein J in human aging and cancer. The international journal of biochemistry & cell biology. 2002; 34(11):1430-48. Epub 2002 Aug. 30. PubMed PMID: 12200037.

37. Nishida K, Adachi W, Shimizu-Matsumoto A, Kinoshita S, Mizuno K, Matsubara K, et al. A gene expression profile of human corneal epithelium and the isolation of human keratin 12 cDNA. Investigative ophthalmology & visual science. 1996; 37(9):1800-9. Epub 1996 Aug. 1. PubMed PMID: 8759347.

38. Nakamura T, Nishida K, Dota A, Kinoshita S. Changes in conjunctival clusterin expression in severe ocular surface disease. Investigative ophthalmology & visual science. 2002; 43(6):1702-7. Epub 2002 May 31. PubMed PMID: 12036968.

39. Li N, Wang N, Zheng J, Liu X M, Lever O W, Erickson P M, et al. Characterization of human tear proteome using multiple proteomic analysis techniques. Journal of proteome research. 2005; 4(6):2052-61. Epub 2005 Dec. 13. doi: 10.1021/pr0501970. PubMed PMID: 16335950.

40. Karnati R, Laurie D E, Laurie G W. Lacritin and the tear proteome as natural replacement therapy for dry eye. Experimental eye research. 2013; 117:39-52. Epub 2013 Jun. 19. doi: 10.1016/j.exer.2013.05.020. PubMed PMID: 23769845; PubMed Central PMCID: PMC3844047.

41. Zhou L, Zhao S Z, Koh S K, Chen L, Vaz C, Tanavde V, et al. In-depth analysis of the human tear proteome. Journal of proteomics. 2012; 75(13):3877-85. Epub 2012 May 29. doi: 10.1016/j.jprot.2012.04.053. PubMed PMID: 22634083.

42. De Paiva C S, Corrales R M, Villarreal A L, Farley W, Li D Q, Stern M E, et al. Apical corneal barrier disruption in experimental murine dry eye is abrogated by methylprednisolone and doxycycline. Investigative ophthalmology & visual science. 2006; 47(7):2847-56. Epub 2006 Jun. 27. doi: 10.1167/iovs.05-1281. PubMed PMID: 16799024.

43. Gipson I K, Spurr-Michaud S, Argueso P, Tisdale A, Ng T F, Russo C L. Mucin gene expression in immortalized human corneal-limbal and conjunctival epithelial cell lines. Investigative ophthalmology & visual science. 2003; 44(6):2496-506. Epub 2003 May 27. PubMed PMID: 12766048.

44. Dursun D, Wang M, Monroy D, Li D Q, Lokeshwar B L, Stern M E, et al. A mouse model of keratoconjunctivitis sicca. Investigative ophthalmology & visual science. 2002; 43(3):632-8. Epub 2002 Feb. 28. PubMed PMID: 11867577.

45. Villareal A L, Farley W, Pflugfelder S C. Effect of topical ophthalmic epinastine and olopatadine on tear volume in mice. Eye Contact Lens. 2006; 32(6):272-6. doi: 10.1097/011c1.0000224360.10319.bl. PubMed PMID: 17099387.

46. Li Z, Woo J M, Chung S W, Kwon M Y, Choi J S, Oh H J, et al. Therapeutic effect of topical adiponectin in a mouse model of desiccating stress-induced dry eye. Investigative ophthalmology & visual science. 2013; 54(1):155-62. Epub 2012 Dec. 6. doi: 10.1167/iovs.12-10648. PubMed PMID: 23211823.

47. Zeger S L, Liang K Y. Longitudinal data analysis for discrete and continuous outcomes. Biometrics. 1986; 42(1):121-30. PubMed PMID: 3719049.

48. Zeger S L, Liang K Y, Albert P S. Models for longitudinal data: a generalized estimating equation approach. Biometrics. 1988; 44(4):1049-60. PubMed PMID: 3233245.

49. Thomas-Tikhonenko A, Viard-Leveugle I, Dews M, Wehrli P, Sevignani C, Yu D, et al. Myc-transformed epithelial cells down-regulate clusterin, which inhibits their growth in vitro and carcinogenesis in vivo. Cancer research. 2004; 64(9):3126-36. Epub 2004 May 6. PubMed PMID: 15126350.

50. Xiao B, Wang Y, Reinach P S, Ren Y, Li J, Hua S, et al. Dynamic ocular surface and lacrimal gland changes induced in experimental murine dry eye. PloS one. 2015; 10(1):e0115333. doi: 10.1371/journal.pone.0115333. PubMed PMID: 25590134; PubMed Central PMCID: PMC4295848.

51. Stewart E M, Aquilina J A, Easterbrook-Smith S B, Murphy-Durland D, Jacobsen C, Moestrup S, et al. Effects of glycosylation on the structure and function of the extracellular chaperone clusterin. Biochemistry. 2007; 46(5):1412-22. Epub 2007 Jan. 31. doi: 10.1021/bi062082v. PubMed PMID: 17260971.

52. Argueso P, Panjwani N. Focus on molecules: galectin-3. Experimental eye research. 2011; 92(1):2-3. Epub 2010 Nov. 30. doi: 10.1016/j.exer.2010.11.009. PubMed PMID: 21111733; PubMed Central PMCID: PMC3034303.

53. Mauris J, Mantelli F, Woodward A M, Cao Z, Bertozzi C R, Panjwani N, et al. Modulation of ocular surface glycocalyx barrier function by a galectin-3 N-terminal deletion mutant and membrane-anchored synthetic glycopolymers. PloS one. 2013; 8(8):e72304. Epub 2013 Aug. 27. doi: 10.1371/j ournal.pone.0072304. PubMed PMID: 23977277; PubMed Central PMCID: PMC3747151.

54. Matani P, Sharrow M, Tiemeyer M. Ligand, modulatory, and co-receptor functions of neural glycans. Frontiers in bioscience : a journal and virtual library. 2007; 12:3852-79. Epub 2007 May 9. PubMed PMID: 17485343.

55. Ochieng J, Fridman R, Nangia-Makker P, Kleiner D E, Liotta L A, Stetler-Stevenson W G, et al. Galectin-3 is a novel substrate for human matrix metalloproteinases-2 and -9. Biochemistry. 1994; 33(47):14109-14. Epub 1994 Nov. 29. PubMed PMID: 7947821.

56. Ulmer T A, Keeler V, Andre S, Gabius H J, Loh L, Laferte S. The tumor-associated antigen 90K/Mac-2-binding protein secreted by human colon carcinoma cells enhances extracellular levels of promatrilysin and is a novel substrate of matrix metalloproteinases-2, -7 (matrilysin) and -9: Implications of proteolytic cleavage. Biochimica et biophysica acta. 2010; 1800(3):336-43. Epub 2009 Aug. 12. doi: 10.1016/j.bbagen.2009.07.030. PubMed PMID: 19665518.

57. Ochieng J, Green B, Evans S, James O, Warfield P. Modulation of the biological functions of galectin-3 by matrix metalloproteinases. Biochimica et biophysica acta. 1998; 1379(1):97-106. Epub 1998 Feb. 19. PubMed PMID: 9468337.

58. Uchino Y, Mauris J, Woodward A M, Dieckow J, Amparo F, Dana R, et al. Alteration of galectin-3 in tears of patients with dry eye disease. Am J Ophthalmol. 2015; 159(6):1027-35 e3. doi: 10.1016/j.ajo.2015.02.008. PubMed PMID: 25703476; PubMed Central PMCID: PMC4426220.

59. Navab M, Anantharamaiah G M, Reddy S T, Van Lenten B J, Buga G M, Fogelman AM. Peptide Mimetics of Apolipoproteins Improve HDL Function. J Clin Lipidol. 2007; 1(2):142-7. doi: 10.1016/j jac1.2007.03.002. PubMed PMID: 18449337; PubMed Central PMCID: PMC2130772.

60. Handattu S P, Garber D W, Horn D C, Hughes D W, Berno B, Bain A D, et al. ApoA-I mimetic peptides with differing ability to inhibit atherosclerosis also exhibit differences in their interactions with membrane bilayers. J Biol Chem. 2007; 282(3):1980-8. doi: 10.1074/jbc.M606231200. PubMed PMID: 17114186.

61. Chieze L, Bolanos-Garcia V M, Pinot M, Desbat B, Renault A, Beaufils S, et al. Fluid and condensed ApoA-I/phospholipid monolayers provide insights into ApoA-I membrane insertion. Journal of molecular biology. 2011; 410(1):60-76. doi: 10.1016/j jmb.2011.04.006. PubMed PMID: 21510960.

62. Lee R C, River LP, Pan FS, Ji L, Wollmann R L. Surfactant-induced sealing of electropermeabilized skeletal muscle membranes in vivo. Proc Natl Acad Sci USA. 1992; 89(10):4524-8. PubMed PMID: 1584787; PubMed Central PMCID: PMC49115.

63. Maskarinec S A, Hannig J, Lee R C, Lee K Y. Direct observation of poloxamer 188 insertion into lipid monolayers. Biophys J. 2002; 82(3):1453-9. doi: 10.1016/S0006-3495(02)75499-4. PubMed PMID: 11867460; PubMed Central PMCID: PMC1301946.

64. Adisetiyo H, Liang M, Liao C P, Aycock-Williams A, Cohen M B, Xu S, et al. Loss of survivin in the prostate epithelium impedes carcinogenesis in a mouse model of prostate adenocarcinoma. PloS one. 2013; 8(7):e69484. Epub 2013 Aug. 13. doi: 10.1371/journal.pone.0069484. PubMed PMID: 23936028; PubMed Central PMCID: PMC3729965.

65. Liang J, Tong P, Zhao W, Li Y, Zhang L, Xia Y, et al. The REST gene signature predicts drug sensitivity in neuroblastoma cell lines and is significantly associated with neuroblastoma tumor stage. International journal of molecular sciences. 2014; 15(7):11220-33. doi: 10.3390/ijms150711220. PubMed PMID: 24968265; PubMed Central PMCID: PMC4139778.

66. Matveev V, Wang X J. Implications of all-or-none synaptic transmission and short-term depression beyond vesicle depletion: a computational study. The Journal of neuroscience: the official journal of the Society for Neuroscience. 2000; 20(4):1575-88. Epub 2000 Feb. 9. PubMed PMID: 10662847.

67. Kierzek A M, Zhou L, Wanner B L. Stochastic kinetic model of two component system signalling reveals all-or-none, graded and mixed mode stochastic switching responses. Molecular bioSystems. 2010; 6(3):531-42. Epub 2010 Feb. 23. doi: 10.1039/b906951h. PubMed PMID: 20174681.

68. Liu T, Yamaguchi Y, Shirasaki Y, Shikada K, Yamagishi M, Hoshino K, et al. Single-Cell Imaging of Caspase-1 Dynamics Reveals an All-or-None Inflammasome Signaling Response. Cell reports. 2014. Epub 2014 Aug. 16. doi: 10.1016/j.celrep.2014.07.012. PubMed PMID: 25127135.

69. Tsianou M, Fajalia A I. Cyclodextrins and Surfactants in Aqueous Solution above CMC: Where are the Cyclodextrins Located? Langmuir: the ACS journal of surfaces and colloids. 2014. Epub 2014 Aug. 16. doi: 10.1021/1a5013999. PubMed PMID: 25126838.

70. Collard M W, Griswold M D. Biosynthesis and molecular cloning of sulfated glycoprotein 2 secreted by rat Sertoli cells. Biochemistry. 1987; 26(12):3297-303. Epub 1987 Jun. 16. PubMed PMID: 3651384.

71. Hochgrebe T, Pankhurst G J, Wilce J, Easterbrook-Smith S B. pH-dependent changes in the in vitro ligand-binding properties and structure of human clusterin. Biochemistry. 2000; 39(6):1411-9. Epub 2000 Feb. 24. PubMed PMID: 10684622.

72. Crabb J W, Miyagi M, Gu X, Shadrach K, West K A, Sakaguchi H, et al. Drusen proteome analysis: an approach to the etiology of age-related macular degeneration. Proceedings of the National Academy of Sciences of the United States of America. 2002; 99(23):14682-7. Epub 2002 Oct. 23. doi: 10.1073/pnas.222551899. PubMed PMID: 12391305; PubMed Central PMCID: PMC137479.

73. Nishida K, Quantock A J, Dota A, Choi-Miura N H, Kinoshita S. Apolipoproteins J and E co-localise with amyloid in gelatinous drop-like and lattice type I corneal dystrophies. The British journal of ophthalmology. 1999; 83(10):1178-82. Epub 1999 Sep. 30. PubMed PMID: 10502582; PubMed Central PMCID: PMC1722813.

74. Calero M, Rostagno A, Matsubara E, Zlokovic B, Frangione B, Ghiso J. Apolipoprotein J (clusterin) and Alzheimer's disease. Microscopy research and technique. 2000; 50(4):305-15. Epub 2000 Aug. 11. doi: 10.1002/1097-0029(20000815)50:4<305::AID-JEMT10>3.0.CO;2-L. PubMed PMID: 10936885.

75. Kounnas M Z, Loukinova E B, Stefansson S, Harmony J A, Brewer B H, Strickland D K, et al. Identification of glycoprotein 330 as an endocytic receptor for apolipoprotein J/clusterin. The Journal of biological chemistry. 1995; 270(22):13070-5. Epub 1995 Jun. 2. PubMed PMID: 7768901.

76. Leeb C, Eresheim C, Nimpf J. Clusterin is a ligand for apolipoprotein E receptor 2 (ApoER2) and very low density lipoprotein receptor (VLDLR) and signals via the Reelin-signaling pathway. The Journal of biological chemistry. 2014; 289(7):4161-72. Epub 2014 Jan. 2. doi: 10.1074/jbc.M113.529271. PubMed PMID: 24381170; PubMed Central PMCID: PMC3924281.

77. Zhang H, Kim J K, Edwards C A, Xu Z, Taichman R, Wang C Y. Clusterin inhibits apoptosis by interacting with activated Bax. Nature cell biology. 2005; 7(9):909-15. doi: 10.1038/ncb1291. PubMed PMID: 16113678.

78. Santilli G, Aronow B J, Sala A. Essential requirement of apolipoprotein J (clusterin) signaling for IkappaB expression and regulation of NF-kappaB activity. The Journal of biological chemistry. 2003; 278(40):38214-9. doi: 10.1074/jbc.C300252200. PubMed PMID: 12882985.

79. Bandamwar K L, Papas E B, Garrett Q. Fluorescein staining and physiological state of corneal epithelial cells. Contact lens & anterior eye: the journal of the British Contact Lens Association. 2014; 37(3):213-23. doi: 10.1016/j.clae.2013.11.003. PubMed PMID: 24332360.

80. Shim Y J, Kang B H, Jeon H S, Park I S, Lee K U, Lee I K, et al. Clusterin induces matrix metalloproteinase-9 expression via ERK1/2 and PI3K/Akt/NF-kappaB pathways in monocytes/macrophages. Journal of leukocyte biology. 2011; 90(4):761-9. Epub 2011 Jul. 12. doi: 10.1189/jlb.0311110. PubMed PMID: 21742938.

81. Radziwon-Balicka A, Santos-Martinez M J, Corbalan J J, O'Sullivan S, Treumann A, Gilmer J F, et al. Mechanisms of platelet-stimulated colon cancer invasion: role of clusterin and thrombospondin 1 in regulation of the P38MAPK-MMP-9 pathway. Carcinogenesis. 2014; 35(2):324-32. Epub 2013 Oct. 3. doi: 10.1093/carcin/bgt332. PubMed PMID: 24085798.

82. Fini M E, Cook J R, R. M, Brinckerhoff C E. Regulation of matrix metalloproteinase gene expression. In: W. P, Mecham R, editors. Matrix Metalloproteinases: Academic Press; 1998.

83. Kompella U B, Kadam R S, Lee V H. Recent advances in ophthalmic drug delivery. Therapeutic delivery. 2010; 1(3):435-56. Epub 2011 Mar. 15. doi: 10.4155/TDE.10.40. PubMed PMID: 21399724; PubMed Central PMCID: PMC3051398.

84. Urtti A. Challenges and obstacles of ocular pharmacokinetics and drug delivery. Advanced drug delivery reviews. 2006; 58(11):1131-5. Epub 2006 Nov. 14. doi: 10.1016/j.addr.2006.07.027. PubMed PMID: 17097758.

85. Dabbs R A, Wilson M R. Expression and purification of chaperone-active recombinant clusterin. PloS one. 2014; 9(1):e86989. Epub 2014 Jan. 28. doi: 10.1371/journal.pone.0086989. PubMed PMID: 24466307; PubMed Central PMCID: PMC3900688.

86. Williamson J F, Huynh K, Weaver M A, Davis R M. Perceptions of dry eye disease management in current clinical practice. Eye & contact lens. 2014; 40(2):111-5. Epub 2014 Feb. 11. doi: 10.1097/ICL.0000000000000020. PubMed PMID: 24508770.

87. Novack G D. Why aren't there more pharmacotherapies for dry eye? Ocul Surf. 2014; 12(3):227-30. doi: 10.1016/j.jtos.2014.05.001. PubMed PMID: 24999105.

88. Sullivan D A, Hammitt K M, Schaumberg D A, Sullivan B D, Begley C G, Gjorstrup P, et al. Report of the TFOS/ARVO Symposium on global treatments for dry eye disease: an unmet need. The ocular surface. 2012; 10(2):108-16. Epub 2012 Apr. 10. doi: 10.1016/j.jtos.2012.02.001. PubMed PMID: 22482471.

What is claimed is:

1. A method of treating dry eye disease comprising:
administering to a patient in need thereof an effective amount of a pharmaceutical composition comprising an isolated clusterin or an isolated protein substantially the same as clusterin, wherein an amount of the pharmaceutical composition immediately below the effective amount of the pharmaceutical composition has substantially no beneficial effect in treating dry eye disease.

2. The method of claim 1, wherein the pharmaceutical composition comprises secreted clusterin.

3. The method of claim 1, wherein the administration is topical.

4. The method of claim 1, wherein the pharmaceutical composition further comprises a liquid carrier, and administration is by contacting the pharmaceutical composition to the surface of an eye of the patient.

5. The method of claim 1, wherein the pharmaceutical composition further comprises a carrier.

6. The method of claim 5, wherein the carrier is a sterile solution.

7. The method of claim 1, wherein the clusterin is recombinant human clusterin.

8. A method of sealing and protecting the ocular surface barrier comprising:
administering to a patient in need thereof an effective amount of a pharmaceutical composition comprising an isolated clusterin or an isolated protein substantially the same as clusterin, wherein an amount of the pharmaceutical composition immediately below the effective amount of the pharmaceutical composition has substantially no beneficial effect in sealing the ocular surface barrier.

9. The method of claim 8, wherein the pharmaceutical composition comprises secreted clusterin.

10. The method of claim 8, wherein the administration is topical.

11. The method of claim 8, wherein the pharmaceutical composition further comprises a liquid carrier, and administration is by contacting the pharmaceutical composition to the surface of an eye of the patient.

12. The method of claim 8, wherein the pharmaceutical composition further comprises a carrier.

13. The method of claim 12, wherein the carrier is a sterile solution.

14. The method of claim 8, wherein the clusterin is recombinant human clusterin.
